(19) United States
(12) Patent Application Publication
(10) Pub. No.: US 2026/0324976 A1
(43) Pub. Date: Oct. 22, 2026

(54) Apparatus, Systems, and Methods for Fluorescence Imaging on Planar Sensors

(71) Applicant: The Regents of the University of California, Oakland, CA (US)

(72) Inventors: Moshiur M. Anwar, San Francisco, CA (US); Micah Roschelle, San Francisco, CA (US)

(21) Appl. No.: 19/115,566

(22) PCT Filed: Sep. 26, 2023
(86) PCT No.: PCT/US2023/033734

§ 371 Date: Mar. 26, 2025

Related U.S. Application Data
(60) Provisional application No. 63/410,557, filed on Sep. 27, 2022.
(60) Provisional application No. 63/410,853, filed on Sep. 28, 2022.

(51) Int. Cl. A61B 5/00; G01N 21/64

(52) U.S. Cl. CPC A61B 5/0071; G01N 21/6456

(57) ABSTRACT

Disclosed herein is a system and methods for imaging tissue. Such systems can be used for in vivo or ex vivo imaging. In vivo imaging can include an imager and an optical front-end. Such front-ends can include a collimator and a filter. The combination can be used to reduce interference or noise coming from oblique light. Additional systems can be used for laparoscopic imaging and/or image-guided surgery, such as tumor resection.

### CROSS-REFERENCE TO RELATED APPLICATIONS

The current application claims priority to U.S. Provisional Patent Application No. 63/410,557, filed Sep. 27, 2022, entitled “Apparatus, Systems, and Methods for Fluorescence Imaging on Planar Sensors” to Anwar, et al. and U.S. Provisional Patent Application No. 63/410,853, filed Sep. 28, 2022, entitled “Apparatus, Systems, and Methods for Fluorescence Imaging on Planar Sensors” to Anwar, et al.; the disclosures of which are hereby incorporated by reference in their entireties.

### STATEMENT REGARDING FEDERALLY SPONSORED RESEARCH OR DEVELOPMENT

This invention was made with Government support under contract DP2DE030713 awarded by the National Institutes of Health. The Government has certain rights in the invention.

### FIELD OF THE INVENTION

The disclosed technology relates generally to lens-less (e.g., lens free) and chip-based fluorescence imagers, including examples of the devices, methods, and design principles for an intraoperative fluorescence imaging system for visualizing microscopic disease. Examples also include lab-on-chip sensors, planar imagers and imaging arrays, DNA, RNA, protein microarrays, ELISA, and PCR-based diagnostics. Examples include implantable imagers and sensors. Examples also include high throughput cell imagers. This has broad implications in the implementation of multi-color (multi-target or multiplexed) and high-performance miniaturized fluorescence imaging systems. Furthermore, this has applications across biomedical applications including, but not limited to, lab on chips, intraoperative imaging, and implantable imagers and sensors. The applications have particular implications in the minimally disruptive diagnosis, monitoring, and treatment of a variety of diseases.

### BACKGROUND

A successful tumor resection surgery results in complete removal of all bulk and microscopic cancer with minimal damage to neighboring healthy tissue. However, microscopic diseased and healthy tissues remain difficult to distinguish intraoperatively, leading many surgical procedures to fall short of this goal. Positive surgical margins (PSMs)—where cancer cells are detected at the edge of the excised tissue post-operation, indicating an incomplete resection—significantly increase risk of recurrence and mortality and require adjuvant treatment, incurring additional costs and burden to the patient, latrogenic damage to tumor-adjacent nerves also remains a persistent problem in certain resections.

Intraoperative imaging during cancer surgeries, of both microscopic diseased and healthy tissue, can help ensure successful removal of all tumor foci with minimal damage to neighboring healthy structures. However, current laparoscopic fluorescence imagers are only capable of macroscopic (millimeter plus scale) visualization and have seen limited application in multiplexed imaging. Surgical microscopes, in contrast, are high resolution, but use bulky optics incompatible with minimally invasive surgical techniques. Thus, there is a need for imaging systems and devices that can provide high-resolution imaging with minimal disruption to the surgical workflow.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, a system for fluorescence imaging includes an optical front-end including a first layer of an optical filter and a second layer of a collimator.

In additional embodiments, the optical front-end comprises a plurality of layers of alternating layers of materials with different refractive indices to create a long pass, short pass, band-pass, notch, or multiband pass interference filter, which may include an absorption filter. In certain embodiments, the optical front-end is affixed to an imaging sensor comprising an array of pixels and wherein the system further comprises a visualizations system in electrical communication with the imaging chip. The imaging sensor may be a CMOS sensor. The front-end may be of a thickness of less than or equal to 5 mm, 2.5 mm, 1.25 mm, 500 μm, 250 μm, and/or 150 μm. The collimator can be angle-selective, where the collimator has 2, 3, 4, 5, or 6 orders of magnitude or greater rejection at 30 degrees or greater off axis. The collimator can be a parallel-hole collimator, a fiber optic plate, an absorptive material surrounding an array of optical fibers, an absorptive material surrounding one or more holes filled with a transparent material, or an absorptive material surrounding holes that are either filled with air or in a vacuum. In certain embodiments the angle-selective device, including but not limited to a parallel-hole collimator or fiber optic plate, may be microfabricated in the layers comprising the image sensor design utilizing metal or active silicon layers as well as specialized post-processing steps. The imaging chip is operationally integrated with a surgical tool, which can be a periscopic probe or a laparoscopic robotic instrument. The imaging chip can include one or more of the following: at least one LED or at least one laser diode light source, is operationally integrated with an implantable imager, or is operationally integrated with a lab-on a chip. Labs-on a chip can be a microarray of DNA, RNA, proteins, cells, tissue, or any biological sample or a diagnostic assay, including a PCR test, ELISA, or lateral flow assay. The imaging chip can also be lens-free and/or use machine learning to improve image quality. A sample being imaged can be one or more of diseased tissue and cancerous tissue. A labeling agent being used can be an antibody, an antibody mimetic, nanobody, a peptide, a peptoid, an aptamer, or a small molecule ligand that selectively binds to the cellular, protein, DNA, RNA, molecular or chemical marker of interest. A cellular marker of interest can be one or more of a tumor-specific antigen, a tumor-associated antigen, an immune-cell-specific antigen, and an immune activation marker. A sample can be imaged with at least two different fluorophore conjugates, where each fluorophore conjugate includes a different fluorophore that emits fluorescent light at a different emission wavelength, and where each fluorophore conjugate comprises a different binding agent that selectively binds to a different marker.

Additional embodiments include a method for imaging a biological sample, including applying a marker to a tissue, and obtaining an image of the tissue using a system, such as described above.

Further embodiments include resecting the tissue to remove diseased tissue from the marked tissue and/or illuminating the tissue with a light source. In some embodiments, obtaining an image of the tissue includes contacting the optical front-end to the tissue. Additionally, the marker can be a fluorescent dye selected from SYBR green, SYBR gold, CAL Fluor Gold 540, CAL Fluor Orange 560, CAL Fluor Red 590, CAL Fluor Red 610, CAL Fluor Red 635, Quasar 570, Quasar 670, Quasar 705, fluorescein, fluorescein isothiocyanate, hexachlorofluorescein, 4′,6-diamidino-2-phenylindole, Hoechst, rhodamine, carboxy-X-rhodamine, and combinations thereof, or the marker is a fluorescent probe comprising a binding agent and a fluorophore. The fluorophore can be selected from Alexa Fluor 488, Alexa Fluor 546, Alexa Fluor 555, Alexa Fluor 594, Alexa Fluor 647, Alexa Fluor 784, Cy3, Cy3.5, Cy5, Cy5.5, and Cy7, and the binding moiety can be selected from a carbohydrate, a lipid, a peptide, a nanobody, a nucleic acid, a protein, and a small molecule.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a conceptual diagram of an example composite filter, in accordance with various embodiments.

FIGS. 2A-2D illustrate aspects of composite filters, in accordance with various embodiments.

FIGS. 3A-3B provide conceptual diagrams of versatile image sensors for intraoperative navigation, in accordance with various embodiments.

FIG. 4 illustrates an example of an implantable imaging device, in accordance with various embodiments.

FIGS. 5A-5C illustrate conceptual diagrams of various composite filters, in accordance with various embodiments.

FIGS. 6A-6C illustrate exemplary data regarding various composite filters, in accordance with various embodiments.

FIGS. 7A-7D provide graphs illustrating example characterizations of an optical front-end designed, in accordance with various embodiments.

FIGS. 8A-8E provide illustrations of example resolution measurements of an optical front-end designed, in accordance with various embodiments.

FIG. 9 is an image illustrating various fascia on and near a prostate, in accordance with various embodiments.

FIGS. 10A-10C provide examples of ex vivo imaging of resected prostate tissue using an optical front-end designed, in accordance with various embodiments.

FIG. 11 provides a conceptual diagram of an example interference filter directly coated on a fiber optic plate (FOP), in accordance with various embodiments.

FIG. 12 provides an example of imaging of PC3-PIP cell cultures, in accordance with the various embodiments.

FIG. 13 provides a graph illustrating exemplary data of signal noise ratios vs. cell cluster sizes, in accordance with the various embodiments.

FIG. 14 provides a diagram of 2D cross-section of geometry used for deriving 2D PSF of a general lens-less imager, in accordance with the various embodiments.

FIG. 15 provides an exemplary model of target and background tissue as a 2D plane with z-axis symmetry, in accordance with various embodiments.

FIGS. 16A-16D provide exemplary data comparing disclosed embodiments with a 15 μm-thick amorphous silicon (a-Si) filter.

### DETAILED DESCRIPTION

Fluorescence contact imagers are provided. Certain embodiments utilize a composite emission filter. Such devices can be lens-less or lens-free, as in they do not utilize lenses. Without lenses, certain instances are smaller than conventional imaging systems while maintaining high resolution. Certain embodiments are used in contact imaging to deliver wide-field of view microscopy while maintaining a thin and planar form factor. Some devices can be used for multiplexed imaging. And further instances are included on laparoscopic and/or guided surgical devices, such that they can be used during minimally-invasive surgery. As a result, many embodiments may be easily integrated on surgical tools with little disturbance to existing surgical workflows.

Before the present invention is described in greater detail, it is to be understood that this invention is not limited to a particular embodiment described, as such may, of course, vary. It is also to be understood that the terminology used herein is for the purpose of describing particular embodiments only, and is not intended to be limiting, since the scope of the present invention will be limited only by the appended claims.

All publications mentioned herein are incorporated herein by reference to disclose and describe the methods and/or materials in connection with which the publications are cited. The present disclosure is controlling to the extent there is a contradiction between the present disclosure and a publication incorporated by reference.

### Definitions

It must be noted that as used herein and in the appended claims, the singular forms “a”, “an” and “the” include plural referents unless the context clearly dictates otherwise. Thus, for example, refer to “an electrode” includes plurality of such electrodes and reference to “the well” includes reference to one or more wells and equivalents thereof known to those skilled in the art, and so forth.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of from about “2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e., an upper component is located at a higher elevation than a lower component in a given orientation, but these terms can change if the component is flipped. The terms “inlet” and “outlet” are relative to a fluid flowing through them with respect to a given structure, e.g., a fluid flows through the inlet into the structure and flows through the outlet out of the structure.

The terms “horizontal” and “vertical” are used to indicate direction relative to an absolute reference, i.e., ground level. However, these terms should not be construed to require structures to be absolutely parallel or absolutely perpendicular to each other. For example, a first vertical structure and a second vertical structure are not necessarily parallel to each other. The terms “top” and “bottom” are used to refer to surfaces where the top is always higher than the bottom relative to an absolute reference, i.e., the surface of the earth. The terms “upwards” and “downwards” are also relative to an absolute reference; upwards is always against the gravity of the earth while downwards is always towards the gravity of the earth.

“Comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. Similarly, ranges that include the language “less than” or “greater than” should be considered to have specifically disclosed all the possible sub-ranges, fractions, and individual numerical values from the value given to the extreme. For instance, the language “less than or equal to 25” should be considered to have specifically disclosed sub-ranges such as less than or equal to 20, less than or equal to 15, etc., as well as individual numbers within that range, for example, 1, 2, 3, 14, 15, 21, 22, 24, 25, etc., and decimals and fractions, for example, 1.2, 3.8, 1½, and 4¾. This applies regardless of the breadth of the range.

The term “fiber optic plate” (abbreviated as FOP) refers to optical devices comprised of a bundle of optical fibers. FOPs can transmit light or an image with high efficiency and low distortion, and no focusing distance is required. FOPs can be designed around a numerical aperture and/or ability to be angle-selective. For example, some FOPs can be transmissive at small angles of incidence but highly absorptive at large angles of incidence.

The term “interference filter” refers to an optical filter designed to provide near-total transmittance of one spectral band with strong rejection of adjacent bands, interference filters can be composed of a stack of alternating layers of materials with different refractive indices. By controlling the thickness of the alternating layers, specific wavelengths of light can either constructively or destructively interfere at each interface. Interference filters can have long-pass, short-pass, band-pass, or notch characteristic or any combination of multiple long-pass, short-pass, band-pass, or notch characteristics in different spectral regions.

The term “angle of incidence” (abbreviated as AOI) refers to an angle between a ray incident on a surface and the line perpendicular (at 90° angle) to the surface at the point of incidence, called the normal. The ray can be formed by any waves, such as optical, acoustic, microwave, and X-ray. The angle of incidence at which light is first totally internally reflected is known as the critical angle. The angle of reflection and angle of refraction are other angles related to beams. Surfaces can include (but are not limited to) functional items, such as a FOP, composite filter, in interference filter, a substrate, a sensor (e.g., an imaging sensor), and/or any other functional surface, in addition to non-functional surfaces.

The terms “peptide”, “oligopeptide”, “polypeptide”, and “protein” are used interchangeably herein to refer to a polymer of amino acid residues. The terms also apply to amino acid polymers in which one or more amino acid residues are an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Substantially purified” generally refers to isolation of a substance (compound, protein, nucleic acid, nanoparticles) such that the substance comprises the majority percent of the sample in which it resides. Typically, in a sample, a substantially purified component comprises 50%, preferably 80%-85%, or more preferably 90-95% of the sample. Techniques for purifying substances of interest include, for example, ion-exchange chromatography, affinity chromatography and sedimentation according to density.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma, and include cancers such as, but are not limited to, pancreatic cancer, lung cancer (non-small cell lung cancer, small cell lung cancer), gastric cancer, ovarian cancer, endometrial cancer, colorectal cancer, oral cancer, skin cancer, cholangiocarcinoma, head and neck cancer, breast cancer, ovarian cancer, melanoma, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

A “ligand” or “binding agent” is any molecule that can be used to target a fluorophore to a cell or other target. In certain embodiments, the ligand is a molecule that selectively binds to a target analyte of interest (e.g., cellular marker) with high binding affinity. By high binding affinity is meant a binding affinity of at least about 10-4 M, usually at least about 10-6 M or higher, e.g., 10-9 M or higher. The ligand may be any of a variety of different types of molecules, as long as it exhibits the requisite binding affinity for the target analyte when conjugated to a fluorophore. In certain embodiments, the ligand has medium or even low affinity for its target analyte, e.g., less than about 10-4 M. As such, the ligand may be a small molecule or large molecule ligand. By small molecule ligand is meant a ligand having a size of less than 10,000 daltons, usually ranging in size from about 50 to about 5,000 daltons, and more usually from about 100 to about 1000 daltons in molecular weight. By large molecule is meant a ligand having a size of more than 10,000 daltons in molecular weight.

A small molecule ligand may be any molecule, as well as binding portion or fragment thereof, that is capable of binding with the requisite affinity to the target analyte of interest (e.g., cellular marker). Generally, the small molecule is a small organic molecule that is capable of binding to the target analyte of interest. The small molecule will include one or more functional groups necessary for structural interaction with the target analyte, e.g., groups necessary for hydrophobic, hydrophilic, electrostatic or even covalent interactions. Where the target analyte is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The small molecule will also comprise a region that may be modified and/or participate in conjugation to a fluorophore, without substantially adversely affecting the small molecule's ability to bind to its target analyte.

Small molecule ligands may comprise cyclical carbon or heterocyclic structures and/or aromatic or polyaromatic structures substituted with one or more of the above functional groups. Small molecule ligands may also include organic compounds comprising alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Small molecules may include structures found among biomolecules, including peptides, carbohydrates, fatty acids, vitamins, steroids, purines, pyrimidines, derivatives, structural analogs or combinations thereof.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. When obtained from such libraries, the small molecule employed will have demonstrated some desirable affinity for the protein target in a convenient binding affinity assay. Combinatorial libraries, as well as methods for the production and screening, are described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Small molecule ligands may also include drugs that selectively bind to receptors on cells, including, without limitation, growth factor receptors, receptor tyrosine kinases, receptor protein serine/threonine kinases, G-protein coupled receptors, cytokine receptors, lectin receptors, and folate receptors. For example, anti-cancer drugs that bind to such cellular receptors may be used as ligands to target fluorophores to cancer cells. Exemplary drugs that may be used as ligands to target cancer cells include, without limitation, Acitinib, Afatinib, Axitinib, Erlotinib, Cabozantinib, Crizotinib, Gefitinib, Imatinib, Ibrutinib, Lapatinib, Neovastat, Nilotinib, Pazopanib, Perifosine, Ponatinib, Regorafenib, Sorafenib, Sunitinib, Trametinib, and Vandetenib.

As pointed out, the ligand can also be a large molecule. Of particular interest as large molecule ligands are antibodies, as well as binding fragments and mimetics thereof.

Also suitable for use as binding agents are peptoids and aptamers. The ligand or binding agent may include a domain or moiety that can be covalently attached to a fluorophore without substantially abolishing the binding affinity for its target analyte (e.g., cellular marker).

The term “antibody” encompasses monoclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)2 and F (ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); diabodies, tetrabodies, affibodies, camelid antibodies, humanized antibody molecules (see, e.g., Riechmann et al. (1988) Nature 332:323-327; Verhoeyan et al. (1988) Science 239:1534-1536; and U.K. Patent Publication No. GB 2,276, 169, published 21 Sep. 1994); and, any functional fragments obtained from such molecules, wherein such fragments retain specific-binding properties of the parent antibody molecule.

“Fv” is an antibody fragment which contains an antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although often at a lower affinity than the entire binding site.

“Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv see, for example, Pluckthun, A. in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) on the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Holliger et al., (1993) Proc. Natl. Acad. Sci. USA, 90:6444-6448.

The term “affibody molecule” refers to a molecule that consists of three alpha helices with 58 amino acids and has a molar mass of about 6 kDa. A monoclonal antibody, for comparison, is 150 kDa, and a single-domain antibody, the smallest type of antigen-binding antibody fragment, 12-15 kDa. See, for exemplary details of affibody structures and uses, Orlova, A; Magnusson, M; Eriksson, T L; Nilsson, M; Larsson, B; Hoiden-Guthenberg, I; Widstrom, C; Carlsson, J et al. (2006). “Tumor imaging using a picomolar affinity HER2 binding affibody molecule”, Cancer Res. 66 (8): 4339-48. Exemplary Affibody. Molecules are commercially available from Abcam Corp. Cambridge Mass.

The phrase “specifically (or selectively) binds” with reference to binding of an antibody or other binding agent to an antigen or analyte (e.g., cellular marker such as a tumor-marker or immune activation marker) refers to a binding reaction that is determinative of the presence of the antigen or analyte in a heterogeneous population of proteins and other biologics. Thus, under designated assay conditions, the specified antibodies or other binding agents bind to a particular antigen or analyte at at least two times the background and do not substantially bind in a significant amount to other molecules present in the sample. Specific binding to an antigen or analyte under such conditions may require an antibody or other binding agent that is selected for its specificity for a particular antigen or analyte. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

The term “conjugated” refers to the joining by covalent or noncovalent means of two compounds or agents (e.g., binding agent specific for a tumor marker or immune activation marker conjugated to a fluorophore).

The terms “subject”, “individual” or “patient” are used interchangeably herein and refer to a vertebrate, preferably a mammal. By “vertebrate” is meant any member of the subphylum chordata, including, without limitation, humans and other primates, including nonhuman primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

### Imaging Chip

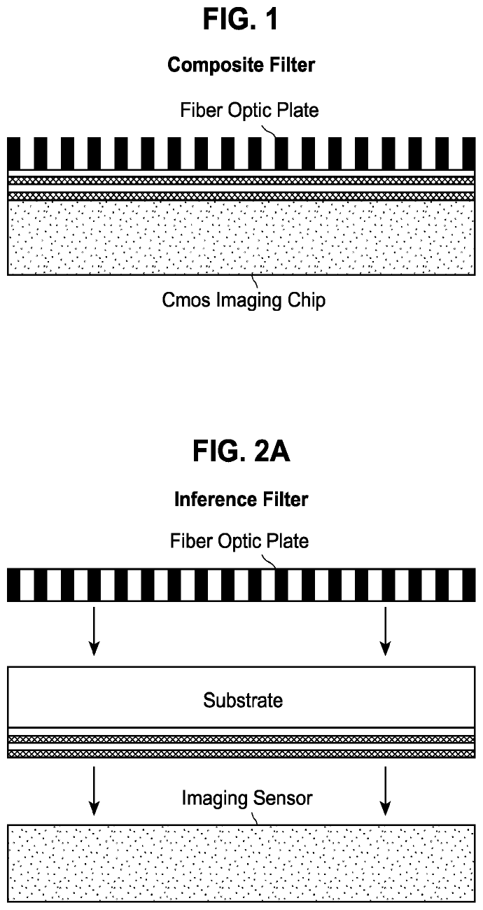

Many embodiments describe a composite emission filter and can provide multiplexed, high-resolution imaging. Such filters can be used in contact imaging, including fluorescence contact imaging. Such devices have uses in surgical imagers and laparoscopic devices, where the smaller size allows minimal invasiveness and minimal interference with a surgical workflow. FIG. 1 is a conceptual diagram of an example composite filter, in accordance with various embodiments described herein. In conventional fluorescence microscopes, multi-layer interference filters are used for this purpose. These filters can be engineered for extremely high performance at any visible and NIR wavelength and can even be made to have multiple passbands for multiplexed imaging. However, the performance degrades rapidly for obliquely incident light. As a result, previous work in lens-less fluorescence imaging has focused on using absorption filters. Here a certain material is chosen that selectively absorbs the excitation light while passing the emissions. These filters are inherently angle-insensitive but suffer from weak performance due to material imperfections.

To compensate for the angle sensitivity of the interference filter, the techniques described herein add a fiber optic plate to the interference filter. This fiber optic plate acts as a collimator and blocks all light beyond a certain angle, compensating for the angle sensitivity of the emission filter. With this approach, the system described herein is able to take advantage of the features of interference filters, including multi-color imaging capabilities. In addition, since the system described herein is imaging without lenses, this fiber-optic plate helps to improve the resolution.

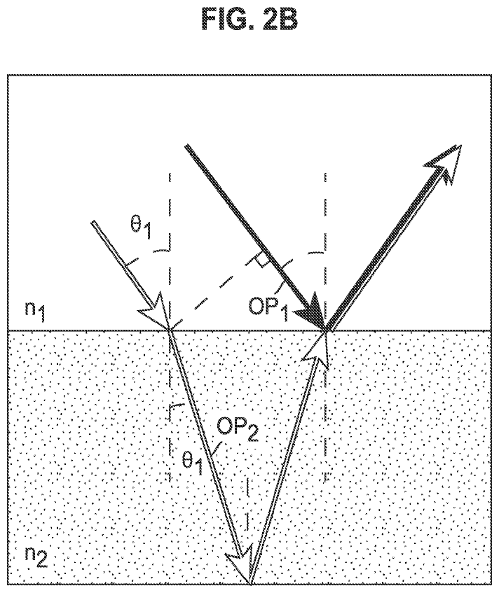

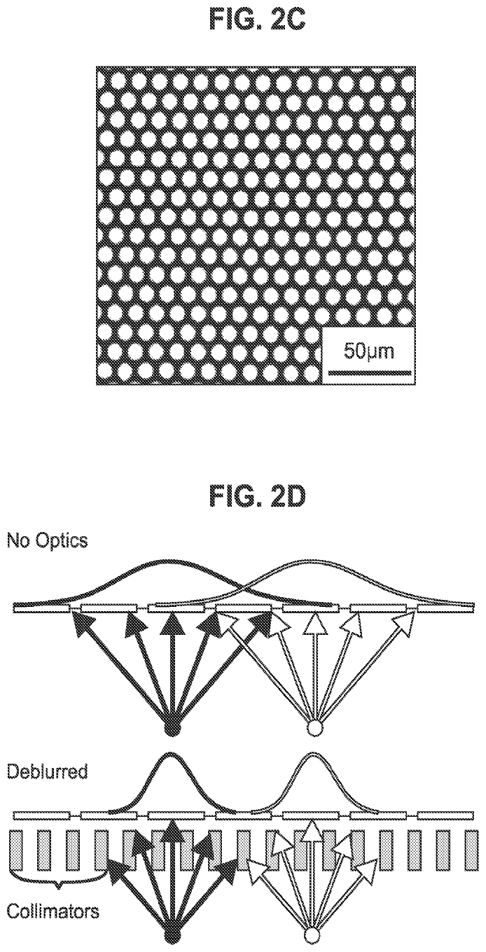

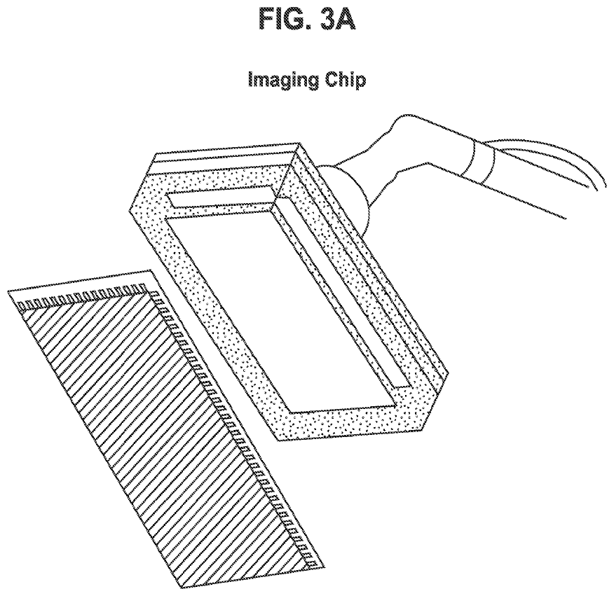

FIG. 2A illustrates an exemplary schematic of a composite filter with an imaging sensor in accordance with many embodiments. In this figure, a fiber optic plate (FOP) and interference filter form a composite emission filter for an imaging sensor. As illustrated, the FOP is distal to the imaging sensor, while the interference filter is proximal to the imaging sensor. However, in some embodiments, the FOP is proximal to the imaging sensor, and the interference filter is distal to the imaging sensor. Additional embodiments utilize multiple interference filters and/or FOPs. Additional details regarding interference filters and FOPs will be discussed further, below. In certain instances, an interference filter can include a substrate, which may provide structure to the interference filter. Such substrates can be any suitable material to provide structure and/or optical throughput. Such substrates include (but are not limited to) fused silica. When using a substrate, the substrate can be of any relevant thickness to provide the proper structure, rigidity, and/or any other property provided by the substrate. In certain circumstances, an interference filter is disposed or deposited directly on a FOP, such as illustrated in FIG. 1. In other circumstances, the interference filter maybe coated or deposited directly on the image sensor or on any another planar surfaces of the system. In further circumstances, the filter may be microfabricated with integrated circuit technology as part of the image sensor design and/or created through post-processing of the image sensor.

In many embodiments, the imaging sensor can be any relevant sensor. In certain instances, the imaging sensor is a Complementary Metal Oxide Semiconductor (CMOS) imaging sensor. Sensors can be an array of pixels with any relevant a number of pixels, size of pixels, and/or pitch of pixels to provide a desired form factor. In certain instances, the array may have dimensions ranging from approximately 24×24 pixels up to approximately 96×96 pixels, and the dimensions do not need to be equal. For example, sensors can have pixels of 24×24 pixels, 24×28 pixels, 24×32 pixels, 24×36 pixels, 24×40 pixels, 24×44 pixels, 24×48 pixels, 24×52 pixels, 24×56 pixels, 24×60 pixels, 24×64 pixels, 24×68 pixels, 24×72 pixels, 36×36 pixels, 36×40 pixels, 36×44 pixels, 36×48 pixels, 36×52 pixels, 36×56 pixels, 36×60 pixels, 36×64 pixels, 36×68 pixels, 36×72 pixels, 36×76 pixels, 36×80 pixels, 36×84 pixels, 36×88 pixels, 36×92 pixels, 36×96 pixels, 48×48 pixels, 48×52 pixels, 48×56 pixels, 48×60 pixels, 48×64 pixels, 48×68 pixels, 48×72 pixels, 48×76 pixels, 48×80 pixels, 48×84 pixels, 48×88 pixels, 48×92 pixels, 48×96 pixels, 60×60 pixels, 60×64 pixels, 60×68 pixels, 60×72 pixels, 60×76 pixels, 60×80 pixels, 60×84 pixels, 60×88 pixels, 60×92 pixels, 60×96 pixels, 72×72 pixels, 72×76 pixels, 72×80 pixels, 72×84 pixels, 72×88 pixels, 72×92 pixels, 72×96 pixels, 88×88 pixels, 88×92 pixels, 88×96 pixels, 92×92 pixels, 92×96 pixels, or 96×96 pixels. It should be noted that the foregoing ranges can be oriented in a longer horizontal or vertical direction, such that an embodiment that is 36×80 pixels is considered equivalent to an array of 80×36 pixels. Pixel size can further be selected from an appropriate size. In various instances, the pixels are 28 μm×28 μm, 32 μm×32 μm, 36 μm×36 μm, 40 μm×40 μm, 44 μm×44 μm, 48 μm×48 μm, 52 μm×52 μm, 56 μm×56 μm, or 60 μm×60 μm. The pitch of pixels (e.g., the distance from one point on a pixel to the equivalent point on an adjacent pixel) can further be adjusted based on manufacturing ability and/or size constraints. Pitch can be selected from approximately 30 μm to approximately 75 μm. It should be noted that pitch cannot be less than a dimension of a pixel, thus pitch can be approximately 30 μm, 35 μm, 40 μm, 45 μm, 50 μm, 55 μm, 60 μm, 65 μm, 70 μm, or 75 μm. It should be noted that a specific embodiment can comprise a pixel array of 80×36 pixels, where each pixel is 44 μm×44 μm with a pitch of 55 μm. It should be noted that the pixel size, pitch, and/or array size can be selected based on available size, availability, manufacturing limitations, and/or any other decision that affects size.

FIG. 2B illustrates an exemplary interface between layers of an interference filter of various embodiments. Interference filters can be fabricated from periodic layers of materials with different refractive indices and precisely tuned thicknesses to cause constructive or destructive interference at each interface for specific wavelengths.

As an angle of incidence (AOI) increases, the optical path length difference between each layer (OP₂−OP₁) decreases, altering the spectral interference of the filter and causing the overall filter response to shift to shorter wavelengths. This phenomenon is illustrated by Equation 1:

λ_{new}=λₚₑₐₖ[1−sin²(θ₁)/n_{eff}]^{1/2} (1)

where θ₁ is the angle of incidence and λₚₑₐₖ and n_{eff} are the peak transmitted wavelength at normal incidence and the effective index of refraction of the filter, respectively. The relation illustrated in Equation 1, shows that for small AOIs the filter response shifts negligibly, maintaining excitation rejection, while for larger AOIs, the shift is magnified, leading to increased bleed-through.

In various embodiments, the interference filter is between approximately 5 μm to 100 μm, such as 5 μm, 10 μm, 15 μm, 20 μm, 25 μm, 30 μm, 35 μm, 40 μm, 45 μm, 50 μm, 60 μm, 70 μm, 75 μm, 80 μm, 90 μm, or 100 μm. Some select embodiments utilize an interference layer that is approximately 10 μm thick. When using a substrate, the substrate may be of any thickness to provide support or structure. Various substrates may be approximately 250 μm to approximately 2 mm, such as 250 μm, 300 μm, 350 μm, 400 μm, 450 μm, 500 μm, 750 μm, 1000 μm, 1250 μm, 1500 μm, 1750 μm, or 2000 μm. Some select embodiments have an interference filter disposed on a substrate of approximately 1 mm (1000 μm).

In some instances, the filter may be a combination of interreference filters and absorption filters. The absorption filter may consist of any absorptive material (that absorbs a specific set of wavelengths and allows the desired emission light to pass through). This material may be organic (such as a dye in a polymer), colored glass, or a semiconductor material that acts as a bandpass, short pass, or long pass filter. One such example is amorphous silicon, which acts as a long pass filter in the near infrared wavelength. Other examples include gallium phosphide, cadmium sulfide, gallium arsenide, indium phosphide, and crystalline silicon.

To compensate for bleed-through from large AOIs, certain embodiments include an angle-selective collimator. Certain embodiments can use a parallel hole collimator to achieve angle-selectivity, while other embodiments use a FOP to achieve angle-selectivity. FOPs of various embodiments include fibers contain a cladding (outer) and core (inner) region with differing refractive indices. The refractive index of the cladding may be chosen to be lower than that of the core such that optical propagation through the fiber is governed by total internal reflection. FOPs allow for large aspect ratios to be achieved without limiting the transmittance of light close to normal incidence. Light incident on the FOP with angles less than the critical angle of the fibers is transmitted through the fibers, while light incidence at angles larger than the critical angle passes through the fibers and into the surrounding absorptive media. This property allows for selectivity characteristics with a sharper cut-off between transmitted ‘collimated’ light and absorbed angled light. The FOP may be composed of fibers combining any materials with a significant refractive index difference surrounded by an absorptive media including but not limited to any of the aforementioned materials. Certain embodiments utilize a low numerical aperture (NA) FOP to provide the angle selectivity. A low-NA FOP allows light transmission at small AOIs while absorbing or otherwise rejecting light with a high AOI. FIG. 2C provides an illustration of an exemplary FOP, where the FOP comprises a matrix of thin optical fibers embedded in a dark, extra-mural absorbing (EMA) glass. For AOIs within the NA of the fibers, the light is guided by total internal reflection through the fiber matrix. As the AOI increases beyond the NA of the fibers, the light is passed through EMA glass experiencing significant attenuation that increases with AOI as the optical pathlength increases. Using optical fibers as the transmissive medium in this way, allows for high aspect ratios necessary for adequate absorption at large AOIs, without significantly reducing the transmittance for AOIs close to normal incidence. In various embodiments, the numerical aperture of the fibers may be (but are not limited to) the range of 0.001-1, such as 0.001, 0.005, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, and/or 1.

As illustrated in FIG. 2C, individual optical fibers are packed into the EMA glass. One parameter in the design of the collimators is the aspect ratio, which is defined as the ratio of the length of the fibers (also referred to as height or depth and refers to a dimension that is perpendicular to the plane of the image) to the diameter of the fibers. A larger aspect ratio, implying that the fibers are significantly longer than their diameter, will result in increased angle selectivity, as angled light will pass through more absorptive material before exiting the collimator. It should be noted that while the fibers can be any shape, including asymmetric, that has a longer length than width. Another parameter is the normal incidence transmittance of the collimator which is determined in part by the fill factor (percentage of surface area that is covered by transmissive fibers). Higher normal incidence transmittance is desired to minimize the insertion loss due to the collimator. However, a smaller fill factor and, hence, lower normal incidence transmittance can increase absorption of angled emissions. The form-factor is to maximize the fill factor and aspect ratio, while limiting the overall thickness of the device.

Fiber size can be between approximately 5 μm and 25 μm, such as 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, or 25 μm. Additionally, packing density and/or pitch are related parameters to demonstrate a number of fibers in a given distance or area-pitch being distance between equivalent points on consecutive fibers, while packing density is number of fibers in a given area. Pitch can be approximately 6 μm and 30 μm, depending on fiber size, manufacturing capabilities, and/or desired pitch. As such, pitch can be 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In the non-limiting example of FIG. 2C, individual fibers possess a diameter of approximately 9 μm and a pitch of approximately 12 μm.

Additionally, circular fibers may be arrayed in any pattern for circle packing, such square, triangular, hexagonal, elongated triangular, trihexagonal, snub square, truncated square, truncated hexagonal, rectitrihexagonal, snub trihexagonal, snub trihexagonal (mirror) truncated trihexagonal, skew quadrilateral, tie kite, isosceles trapezoid, right trapezoid, and/or any other pattern of packing.

As mentioned above, FOP thickness (or other collimator) can be selected for performance and/or for size. In various embodiments, thickness is less than 2 cm, others <1 cm, others <0.5 cm, and others <0.2 cm. In other embodiments, thickness can be between approximately 50 μm and 500 μm, such as 50 μm, 75 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, or 500 μm. Select embodiments utilize a FOP having a thickness of 250 μm.

A FOP can also act as a collimator, such as schematically illustrated in FIG. 2D. Collimators restrict the angle of view of each pixel by blocking light incident at oblique angles, deblurring the image. By acting as a collimator, FOPs can improve imaging resolution by attenuating divergent light that contributes to blur, effectively restricting the field of view (FOV) of each pixel to a smaller area. In lens-less fluorescence contact imaging, for maximum resolution the fluorescent signal from each cell may be isolated to the pixel directly opposite it. However, fluorophores can emit light isotopically. Thus, light emitted at an angle diverges as it travels across the separation distance between an imaging plane and the image sensor, spreading to adjacent pixels and blurring the resulting image. While such an improvement in resolution through comes with a reduction in fluorescence signal seen by the sensor, the background at each pixel due to tissue autofluorescence is also reduced. A noise reduction from autofluorescence is advantageous, because autofluorescence can easily dominate the imager signal, reducing contrast, limiting pixel dynamic range, and increasing signal dependent noise.

FOPs can be fabricated from a combination of any transmissive (core) and absorptive media (surrounding or cladding). The absorptive media may be as absorptive as possible across the entire spectral region in which the sensor is intended to operate, such that the fill factor and aspect ratio constraints can be minimized. Possible absorptive media include either semiconductor substrates or absorptive dyes mixed within a structural material including but not limited to epoxy, glass, silicone, or PDMS. The PDMS or other polymer material can be made with an absorptive dye. Other materials include semiconductor materials (such as a silicon wafer), opaque inorganic materials (also including semiconductors, metals, plastics), organic materials can also be used as the cladding material. The transmissive hole array can be constructed simply as air gaps within the material or can be filled with a media that is optically transmissive in the spectral region of interest including but not limited to epoxy, glass, silicon (for example for NIR), semiconductors transparent to the wavelength of interest, or PDMS.

FOPs may even be fabricated on-chip by patterning semiconductor materials with adequate absorption in the visible region (as cladding material) and that are compatible with common microfabrication techniques. The collimator may also be fabricated by creating pillars of transmissive material with no cladding around them (such that ‘air’ is the cladding) providing a lower index of refraction surrounding area to trap light within the transmissive pillars.

Certain collimators may also utilize optical phenomena such as refraction or interference to achieve similar properties. Such implementations are not limited to but may take the form of micro-lens arrays or diffraction gratings with the characteristics being that they are planar and have a thickness of less than 5 mm and can sufficiently collimate the incident light. In some instances, the thickness can be less than 1 cm.

The selection of a suitable collimator (e.g., FOP) for this optical front-end may depend on the angle selectivity to adequately block angled excitation that may otherwise pass through the interference filter. In some fluorescence imaging applications, the fluorescence emissions are often 4 to 6 orders of magnitude weaker than the excitation background. As such, fluorescence filters in those applications may be capable of providing 4 to 6 orders of magnitude excitation rejection. Alternative applications are conceivable in which this threshold may be lowered to 2 orders of magnitude excitation rejection. These, for example, can be applications with a high fluorescence signal. Additionally, alternative applications are also conceivable in which this threshold may be raised to 10 orders of magnitude excitation rejection. Interference filters are capable of providing this level of rejection at normal incidence, but as described previously with increasing incident angle off the axis perpendicular to the surface of the filter, the filter characteristic shifts to lower wavelengths increasing excitation bleed-through. The precise angle at which the excitation bleed-through exceeds the threshold determined by the level of rejection may be dependent on the proximity of the emission spectra of the excitation source in relation to the cut-off wavelength of the filter and can be determined through measurement. The closer that the excitation wavelength is to the cut-off wavelength of the filter, the lower the angle will be at which rejection provided by the interference filter starts to decay. For angles beyond this angle, the planar collimator may add additional rejection to preserve filtering performance above the determined threshold. For example, if 5 orders of magnitude excitation rejection are required across all angles of incidence, the optical front-end should provide this. For example, if by 30 degrees the rejection of the interference filter decays to 2 orders of magnitude (from 4-6 at normal incidence, where normal is defined as perpendicular to the plane of incidence), the planar collimator provides at least an additional 3 orders of magnitude rejection to reach the threshold.

The aspect ratio (height over width (or diameter)) may influence the system. The higher the aspect ratio, the more selective the collimator plate is (e.g., by allowing only a smaller set of angles of light to pass through). Higher aspect ratios can therefore be made by having a taller substrate, but this increases the form-factor of the device. Thinner collimator plates (i.e. reduced height) can be created by also having a shorter width. However, too small a width begins to reduce the pixel fill factor, as an increased amount of blocking or absorptive material is placed over the photosensitive element (i.e. photodiode) of the imager, blocking more light and reducing signal. Therefore, the system may utilize an adequate fill factor, which sets the lower bound of the width, and an appropriate angle selection, which then defines the height of the layer. Overall, the height (thickness) of the collimator layer may increase the bulk of the overall device. In some instances, the system may utilize 2 orders of magnitude or greater rejection at 30 degrees or greater off axis. In some instances, the system may utilize 3 orders of magnitude or greater rejection at 30 degrees or greater off axis. In some instances, the system may utilize 4 orders of magnitude or greater rejection at 30 degrees or greater off axis. In some instances, the system may utilize 5 orders of magnitude or greater rejection at 30 degrees or greater off axis. In some instances, the system may utilize 6 orders of magnitude or greater rejection at 30 degrees or greater off axis. In some instances, the system may utilize the total optical front-end to be less than 1 cm thick. In some instances, the system may utilize the total optical front-end to be less than 0.5 cm thick. In some instances, the system may utilize the total optical front-end to be less than 0.25 cm thick. In some instances, the system may utilize the total optical front-end to be less than 1 mm thick. In some instances, the system may utilize the total optical front-end to be less than 0.5 mm thick. In some instances, the system may utilize the total optical front-end to be less than 250 microns thick. In some instances, the system may utilize the total optical front-end to be less than 150 microns thick. In certain examples, the thickness of the layer of optical filter material is less than 100 microns. For example, the thickness of the layer of filter material may range from 1 micron to 100 microns, including any thickness within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 microns in thickness. In some cases, the system may utilize the fill factor over the photosensitive element to be greater than 95%. In some cases, the system may utilize the fill factor over the photosensitive element to be greater than 75%. In some cases, the system may utilize the fill factor over the photosensitive element to be greater than 50%. In some cases, the system may utilize the fill factor over the photosensitive element to be greater than 25%. In some cases, the system may utilize the fill factor over the photosensitive element to be greater than 15%. In some instances, the system may utilize the aspect ratio of the holes or fibers (height divided by width (or diameter)) to be greater than or equal to 30. In some instances, the system may utilize the aspect ratio of the holes or fibers (height divided by width (or diameter)) to be greater than or equal to 20. In some instances, the system may utilize the aspect ratio of the holes or fibers (height divided by width (or diameter)) to be greater than or equal to 10. In some instances, the system may utilize the aspect ratio of the holes or fibers (height divided by width (or diameter)) to be greater than or equal to 5. In some instances, the system may utilize the aspect ratio of the holes or fibers (height divided by width (or diameter)) to be greater than or equal to 3.

The disclosed optical front-end can be fabricated by separately manufacturing each of the layers including but not limited to a planar collimator and any number of interference filters and then binding them together with optically transparent binding agent including but not limited to epoxy. Each of the components can be fabricated on a mechanical substrate, such as glass, that has transmission properties compatible with fluorescence imaging (i.e. transparent at all wavelengths of interest.) Through this method the disclosed optical front-end has the advantage that it may be fabricated entirely from commercially available parts, allowing for quick and easy implementation.

Alternatively to reduce the overall thickness of the optical front-end and to reduce imaging artifacts, the interference filters can be coated directly on the planar collimator providing that the planar collimator has a smooth surface and is composed of a material compatible with available material deposition techniques including but not limited to vacuum thermal evaporation, sol-gel technique, chemical bath deposition, spray pyrolysis technique, plating, electroplating, electroless deposition, chemical vapor deposition, low pressure chemical vapor deposition, plasma enhanced chemical vapor deposition, atomic layer deposition, radio frequency sputtering, direct current sputtering, ion plating evaporation, molecular beam epitaxy, arc evaporation, laser beam evaporation, and electron beam evaporation. Following the design guidelines outlined earlier, the interference filter may be coated on either or both sides of the planar collimator.

The disclosed optical front-end has applications in any fluorescence imaging or sensing system which do not primarily rely on lenses. In many instances, lenses are not used. However, micro lenses, phase masks, amplitude masks, or micro lenses arrays of various sizes and arrangements can also be integrated on the optical front-end. These applications include but are by not limited to implantable fluorescence imagers or sensors for in vivo imaging; multiplexed sensing arrays for DNA analysis, protein analysis, PCR, flow cytometry, cell counting, fluorescent bead counting; or small form-factor fluorescence cameras or microscopes for pathological analysis, live cell imaging, or intraoperative imaging.

Additionally, contact sensors (e.g., a combination of a composite filter or “front-end” with an imaging sensor) as described herein can eliminate a need for bulky optical lenses by capturing light emitted from cells directly in contact with the imager surface before the light diverges, can be used in intraoperative imaging. The close proximity of the imager to the tissue increases sensitivity over imagers placed far from the tissue. Taking advantage of the ultra-thin, planar, and array-like structure inherent to CMOS image sensor technology, these imagers can be easily integrated on to the surface of surgical tools without interrupting the surgical workflow and can be scaled to accommodate large fields-of-view while maintaining resolution and maneuverability. Despite these advantages, contact imager designs may use thin and planar alternatives to conventional fluorescence optical front-ends and focusing optics, which has proven a difficult engineering challenge.

Fluorescence optical front-ends play a role in determining imager performance and utilize unconventional designs for on-chip solutions. Due to the small absorption cross-section and optical properties of conventional fluorophores, fluorescence emissions are often 4 to 6 orders of magnitude weaker than the excitation light in intensity and red-shifted only 10-100 nm in wavelength from their absorption peak. As a result, high-performance optical front-ends are used for the detection of weak fluorescence signals from an intense excitation background. In traditional fluorescence microscopes, multi-layer interference filters are used for this purpose as they can be engineered to have high out-of-band rejection, sharp cut-off transitions, and near-total passband transmittance for any visible or near-IR spectral band. However, interference filters are innately angle-sensitive; for progressively oblique incident angles, the filter passband shifts to shorter wavelengths, increasing excitation bleed-through. Consequently, this class of filters has included focusing optics to ensure captured excitation (illumination) and emission light is incident perpendicular to the filter surface. However, in contact imaging schemes the excitation light may be introduced obliquely and there are no collimating lenses to guarantee that all light is normally incident on the filter, precluding the use of interference filters.

### Systems

One noteworthy application of chip-based fluorescence imaging is in intraoperative surgical guidance. A successful tumor resection surgery results in complete removal of all bulk and microscopic cancer with minimal damage to neighboring healthy tissue. However, surgical guidance is largely accomplished by visual examination and palpitation which lacks adequate contrast and sensitivity, leading procedures across many common cancer types to fall short of this goal. Conventional lensed systems face tradeoffs between maneuverability and sensitivity due to the use of optical lenses. Surgical microscopes achieve microscopic resolution but require bulky optical lenses, which limit the maneuverability of the device and obstruct the surgical workflow. To address these shortcomings, laparoscopes have been proposed. In these systems the optical components can be shrunk down to fit through a laparoscopic port and are designed to accommodate a wide field of view. However, these design constraints significantly limit achievable sensitivity of these devices, limiting them to macroscopic detection.

For prostate cancer, specifically, positive surgical margins (PSMs), where cancer cells are detected at the edge of the excised tissue post-operation, indicating an incomplete resection, have been found to occur in more than 20% of prostatectomies, the highest rate for all cancers among men. PSMs significantly increase a risk of recurrence and mortality and require adjuvant treatment, incurring additional costs and burden to the patient, latrogenic damage to tumor-adjacent nerves also remains a persistent problem in prostatectomies. Even in minimally invasive, robot-assisted radical prostatectomies, the prevalence of erectile dysfunction, which is caused by damage to the cavernous nerves, is posited to range from 10-46% at 1-year post-operation.

Among several proposed intraoperative imaging modalities, fluorescence imaging, which relies on targeted fluorescent contrast agents (fluorophores) to distinguish specific tissue types, is an approach. Fluorophores can be conjugated to biological probes, such as antibodies or small molecules, targeted towards specific cellular markers, generating high-contrast fluorescence signals with cellular-level specificity. Moreover, multiplexed imaging of disparate tissue types, for example, tumor and nerves, is possible with different wavelength fluorescent tracers. Some of these imagers have even been integrated into laparoscopic tools compatible with minimally invasive robotic surgery workflows that are becoming standard-of-care for radical prostatectomies. However, such systems are macroscopic imagers incapable of resolving microscopic disease. While intraoperative surgical microscopes have been proposed for high-resolution imaging, they require bulky optics that are difficult to maneuver within the surgical cavity, are only capable of line-of-sight imaging and are incompatible with minimally invasive surgical techniques.

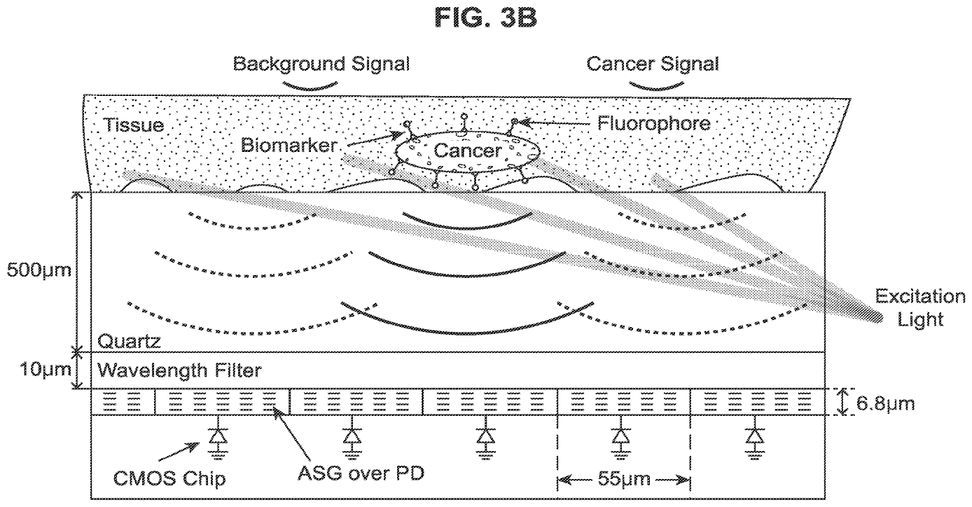

FIGS. 3A-3B provide conceptual diagrams of an example versatile image sensor for intraoperative navigation, in accordance with various techniques described herein. To provide microscopic detection while maintaining maneuverability, the techniques described herein include an intraoperative imaging chip. In this system, all of the bulky optical components are removed and images are captured with just the image sensor itself. By placing the chip directly in contact with the tissue, systems can maintain adequate resolution and high sensitivity by capturing fluorescence emission before they diverge. As a result, this technology is capable of microscopic detection. Also, taking advantage of its small and inherently planar form factor, it is highly maneuverable and can be integrated on existing surgical tools. Two challenges are in maintaining resolution without lenses and integrating the optical front-end necessary for separating the fluorescence signal from the excitation background.

As illustrated in FIG. 3B, various embodiments include a light source to provide an excitation wavelength. The light source can be placed such that it illuminates a target tissue at an oblique angle. The contact sensor of various imaging chips can then intercept normally incident light emitted from markers or dyes on the tissue.

Additional embodiments can include a visualization system in electrical communication with an imaging chip as described herein. The visualization system can be used to observe any visible or fluorescent signal emitted from a surgical device or scope incorporating an imaging chip, as described herein. Such visualization systems can include a monitor (or other display), a fluorometer, a luminometer, and/or any other device for measuring a signal emitted from a sample or tissue. In certain instances, the visualization system is connected to an imaging chip via electronic connections (e.g., wires, cords, etc.) or via a wireless interface, such as ultrasound, RF, low frequency EM, magnetic, etc.

Another notable application is implantable sensors for in vivo imaging, whereby the entire image sensor must fit within the body, or within a tumor, lesion, organ, or portion of tissue. This can include imaging within tissue, or in vivo flow cytometry (for tagged cells, molecules, or nanoparticles), or imaging of genetically engineered cells, such as with CAR-T cells.

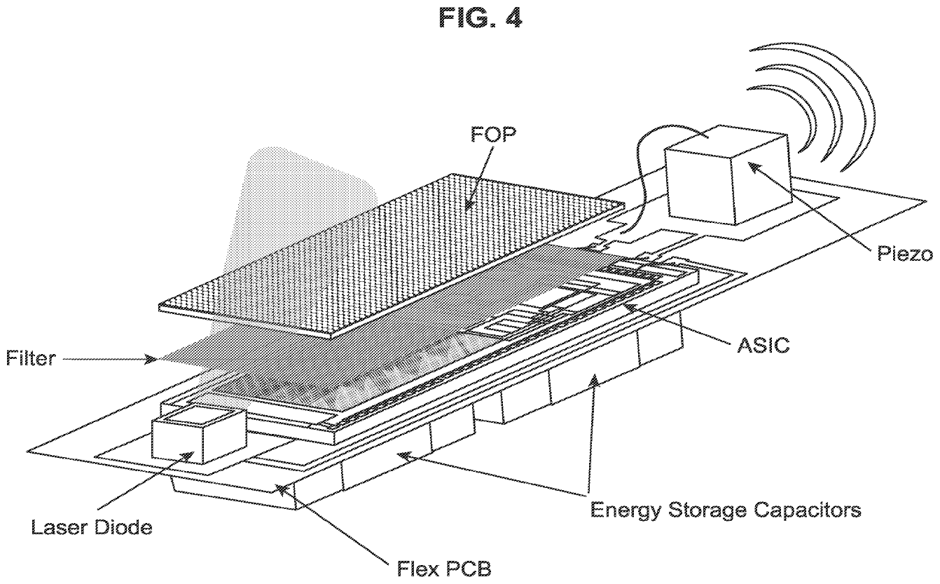

Certain embodiments are directed to implantable imagines, such as illustrated in FIG. 4. The smaller form factor of a lens-less design may be better for implantation. In such embodiments, a FOP and filter can be mounted to an imaging sensor (e.g., a CMOS sensor) and an application specific integrated circuit (ASIC) and printed circuit board (PCB). Additional embodiments include a wireless interface to transmit images to an external device. The wireless interface can be any wireless modality of power and data transfer, including ultrasound, RF, low frequency EM, magnetic.

Power to the implantable device can be provided via capacitors, batteries (e.g., Li-Ion, alkaline, NIMH, and/or other battery type), or other electrical storage device. Such batteries can be charged via any applicable means, such as via motion or pressure (e.g., piezo electric device), wireless (e.g., Qi protocol). A light source, such as a light emitting diode, laser diode, and/or any other applicable light source. Such light sources can be wavelength specific based on inherent emission or by using filters. Such light can be tuned or keyed to an excitation wavelength of a fluorescent marker, such as described herein.

Another notable application is lab-on-chip sensors utilizing fluorescence imaging and sensing. These systems can span from DNA- or RNA-based microarrays to protein microarrays, to cellular or tissue microarrays. This can include point-of-care sensors, and point-of-care antigen- or PCR-based diagnostic assays.

Another notable application is optogenetics, which can include imaging of neuronal activity in the nervous system (central or peripheral). Any other application where a small form-factor fluorescence imager is beneficial is also applicable to this technology. Any other application where a low-cost fluorescence imager is beneficial is also applicable to this technology. Any other application where an arrayable fluorescence imager is beneficial is also applicable to this technology.

In constructing such systems, certain embodiments include a light source. Such a light source can be used as an excitation source for a fluorescent probe or dye. Such probes or dyes can be used to identify tissues of interest, such as cancerous tissue and/or cells. Such light source can be a white light source or a source specific to a particular wavelength, spectrum, and/or range of wavelengths. Wavelengths can be generated by using a specific light source or diode that illuminates a specific wavelength or range of wavelengths. Additional embodiments can utilize one or more filters to provide a wavelength of light. Certain implementations may have a system that allows to change a wavelength or range of wavelengths emitted from the light source and/or utilize multiple light sources, where each light source is capable of providing a single wavelength or range of wavelengths.

Wavelengths or ranges of wavelengths can range from ultraviolet (UV) light to infrared (IR) wavelengths of light. Exemplary ranges include near-UV (~300 nm to ~380 nm), violet (~380 nm to ~440 nm), blue (~440 nm to ~485 nm), cyan (~485 nm to ~510 nm), green (~510 nm to ~565 nm), yellow (~565 nm to ~590 nm), orange (~590 nm to ~625 nm), red (~625 nm to ~740 nm), or near-IR (~740 nm to ~850 nm).

Additional systems can include a computing device for image analysis, feature recognition, and/or any other purpose. Such computing devices can bet set to perform such functions automatically, while others allow user input or settings. Some such systems utilize trained machine learning models. Various machine learning models are capable of improving image quality.

### Methods of Use

Various implementations are directed to methods of using an imaging chip as described herein. In various instances, imaging chips can be used in vivo and/or ex vivo. In vivo imaging includes imaging live tissue within a subject (e.g., animal, mammal, non-human mammal, human, primate, simian, monkey, ape, rodent, mouse, ungulate, etc.), while ex vivo can include imaging tissue samples, biopsies, and/or any other excised tissue sample. Ex vivo samples can be placed imaged on a slide (e.g., microscope slide) or directly from the excised tissue itself.

In certain embodiments, a marker (e.g., dye or probe) can be applied to a tissue, whether in vivo or ex vivo. Such dye or probe can be colorimetric and/or fluorescent. A probe can include a fluorophore or other chromatographic moiety for imaging a cell or tissue type. The chromatographic moiety can be conjugated to an agent that targets a molecule of interest-such agents may be referred to as a binding agent, targeting agent, binding moiety, and/or binding moiety. Such binding agents can include (but are not limited to) a carbohydrate, a lipid, a peptide, a nucleic acid (e.g., RNA, DNA, etc.), a protein, and/or other large or small molecule of interest. Exemplary fluorescent moieties and dyes can include (but are not limited to) SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 488, Alexa Fluor 546, Alexa Fluor 555, Alexa Fluor 594, Alexa Fluor 647, and Alexa Fluor 784, a cyanine dye such as Cy3, Cy3.5, Cy5, Cy5.5, and Cy7, fluorescein, 2′,4′,5′,7′-tetrachloro-4-7-dichlorofluorescein (TET), carboxyfluorescein (FAM), fluorescein isothiocyanate (FITC), 6-carboxy-4′,5′-dichloro-2′,7′-dimethoxyfluorescein (JOE), hexachlorofluorescein (HEX), 4′,6-diamidino-2-phenylindole (DAPI), Hoechst, rhodamine, carboxy-X-rhodamine (ROX), tetramethyl rhodamine (TAMRA), Texas Red, any other fluorescent dye or fluorophore, and combinations thereof. Dyes can further include near-IR dyes or fluorophores, such as IRDye dyes (e.g., IRDye 800CW, IRDye 680RD, IRDye 700, IRDye 750, and IRDye 800RS), CF dyes (e.g., CF680, CF680R, CF750, CF770, and CF790), Tracy dyes (e.g., Tracy 645 and Tracy 652), Alexa dyes (e.g., Alexa Fluor® 660 dye, Alexa Fluor® 700 dye, Alexa Fluor® 750 dye, and Alexa Fluor® 790), cyanine dyes (e.g., Cy7 and Cy7.5), thienothiadiazole dyes, phthalocyanine dyes, squaraine dyes, rhodamine dyes and analogues (e.g., Si-pyronine, Si-rhodamine, Te-rhodamine, and Changsha), borondipyrromethane (BODIPY) dyes, seminaphthofluorone xanthene dyes, benzo[c]heterocycle dyes (e.g., isobenzofuran dyes), and quantum dots. For a review of NIR fluorophores and their use in fluorescence imaging, see, e.g., Escobedo et al. (2010) Curr. Opin. Chem. Biol. 14 (1): 64-70, Hilderbrand et al. (2010) Curr. Opin. Chem. Biol. 14 (1): 71-79, Yuan et al. (2013) Chem. Soc. Rev. 42 (2): 622-661, Vats et al. (2017) Int. J. Mol. Sci. 18 (5), Zhang et al. (2017) Nat. Rev. Clin. Oncol. 14 (6): 347-364, Gao et al. (2010) Curr. Top. Med. Chem. 10 (12): 1147-1157, Zhao et al. (2018) Wiley Interdiscip Rev Nanomed Nanobiotechnol. 10 (3): e1483, and Haque et al. (2017) Bioorg. Med. Chem. 25 (7): 2017-2034; herein incorporated by reference in their entireties.

Fluorescent dyes and probes have respective excitation and emission wavelengths that are known or published for any particular dye or probe. A light source used in certain instances can be keyed or matched to an excitation maximum or excitation spectrum of the dye or probe. Additionally, an interference filter can be keyed or matched to the emission maximum or emission spectrum of the probe and/or dye used. Such wavelengths of light can include light from UV, visible, and/or IR ranges, as described above.

In some embodiments, a fluorophore conjugate is used in fluorescence imaging that comprises a binding agent that selectively binds to a molecule of interest. In some embodiments, multiple fluorophore conjugates are used, wherein the different fluorophore conjugates bind to different molecules of interest. When using multiple probes or dyes, such the molecules of interest may be on cells of the same cell-type or different cell-types. The moiety for targeting can include antibodies, antibody fragments, antibody mimetics, and aptamers as well as small molecules, peptides, peptoids, or ligands that bind selectively to molecules of interest. Antibodies that can be used include (but are not limited to) monoclonal antibodies, polyclonal antibodies, as well as hybrid antibodies, altered antibodies, chimeric antibodies, and humanized antibodies. Antibodies may include hybrid (chimeric) antibody molecules (see, for example, Winter et al. (1991) Nature 349:293-299; and U.S. Pat. No. 4,816,567); F(ab′)2 and F (ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); diabodies, tetrabodies, affibodies, camelid antibodies, humanized antibody molecules (see, e.g., Riechmann et al. (1988) Nature 332:323-327; Verhoeyan et al. (1988) Science 239:1534-1536; and U.K. Patent Publication No. GB 2,276, 169, published 21 Sep. 1994); herein incorporated by reference in their entireties. This additionally includes any functional fragments obtained from such molecules, wherein such fragments retain specific-binding properties of the parent antibody molecule.

In other embodiments, the binding agent comprises an aptamer that specifically binds to the marker of interest. Any type of aptamer may be used, including a DNA, RNA, xeno-nucleic acid (XNA), or peptide aptamer that specifically binds to the tumor antigen. Such aptamers can be identified, for example, by screening a combinatorial library. Nucleic acid aptamers (e.g., DNA or RNA aptamers) that bind selectively to a target tumor antigen can be produced by carrying out repeated rounds of in vitro selection or systematic evolution of ligands by exponential enrichment (SELEX). Peptide aptamers that bind to a marker of interest may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Nucleic Acid Aptamers: Selection, Characterization, and Application (Methods in Molecular Biology, G. Mayer ed., Humana Press, 2016), Aptamers Selected by Cell-SELEX for Theranostics (W. Tan, X. Fang eds., Springer, 2015), Cox et al. (2001) Bioorg. Med. Chem. 9 (10): 2525-2531; Cox et al. (2002) Nucleic Acids Res. 30 (20): e108, Kenan et al. (1999) Methods Mol. Biol. 118:217-231; Platella et al. (2016) Biochim. Biophys. Acta November 16 pii: S0304-4165 (16) 30447-0, and Lyu et al. (2016) Theranostics 6 (9): 1440-1452; herein incorporated by reference in their entireties.

In other embodiments, the binding agent comprises a small molecule ligand. Small molecule ligands encompass numerous chemical classes, e.g., small organic compounds having a molecular weight of less than about 10,000 daltons, less than about 5,000 daltons, or less than about 2,500 daltons. The small molecule will include one or more functional groups necessary for structural interaction with the target analyte, e.g., groups necessary for hydrophobic, hydrophilic, electrostatic or even covalent interactions. Where the target analyte is a protein (e.g., cellular marker), the ligand will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, or preferably at least two of the functional chemical groups. The small molecule may also comprise a region that may be modified and/or participate in conjugation to a fluorophore, without substantially adversely affecting the small molecule's ability to bind to its target analyte.

Small molecule ligands can comprise cyclical carbon or heterocyclic structures and/or aromatic or polyaromatic structures substituted with one or more of the above functional groups. Small molecule ligands may also include organic compounds comprising alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Small molecule ligands are also found among biomolecules including peptides, carbohydrates, fatty acids, steroids, purines, pyrimidines, derivatives, structural analogs or combinations thereof. The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. When obtained from such libraries, the small molecule employed will have demonstrated some desirable affinity for the protein target in a convenient binding affinity assay. Combinatorial libraries, as well as methods for the production and screening, are described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Small molecule ligands may also include drugs that selectively bind to receptors on cells, including, without limitation, growth factor receptors, receptor tyrosine kinases, receptor protein serine/threonine kinases, G-protein coupled receptors, cytokine receptors, lectin receptors, folate receptors, prostate-specific membrane antigen (PSMA), carbonic anhydrase IX receptor, and biotin receptors. For example, anti-cancer drugs that bind to such cellular receptors may be used as ligands to target fluorophores to cancer cells. Exemplary drugs that may be used as ligands to target cancer cells include, without limitation, Acitinib, Afatinib, Axitinib, Erlotinib, Cabozantinib, Crizotinib, Gefitinib, Imatinib, Ibrutinib, Lapatinib, Neovastat, Nilotinib, Pazopanib, Perifosine, Ponatinib, Regorafenib, Sorafenib, Sunitinib, Trametinib, and Vandetenib.

In other embodiments the binding agent comprises a membrane-targeted cleavable probe that becomes activated when it encounters a protease. Such probes comprise a synthetic peptide substrate comprising a protease cleavage site coupled to a fluorophore and a membrane targeting domain. Upon cleavage by a protease, the fluorophore is deposited in cell membranes. For a description of such protease-activated peptide probes, see, e.g., Page et al. (2015) Nature Communications 6 (8448), Backes et al. (2000) Nat. Biotechnol. 18:187-193; herein incorporated by reference.

Fluorophores may be conjugated to binding agents by any suitable method. In some instances, the fluorophore and binding agent may be directly linked, e.g., via a single bond, or indirectly linked e.g., through the use of a suitable linker, e.g., a polymer linker, a chemical linker, or one or more linking molecules or moieties. In some instances, attachment of the fluorophore and binding agent may be by way of one or more covalent interactions. In some instances, the fluorophore or binding agent may be functionalized, e.g., by addition or creation of a reactive functional group. Functionalized fluorophores or binding agents may be modified to contain any convenient reactive functional group for conjugation such as an amine functional group, a carboxylic functional group, a sulfhydryl group, a thiol functional group, and the like.

Any convenient method of bioconjugation may be used including, but not limited to, glutaraldehyde crosslinking, carbodiimide crosslinking, succinimide ester crosslinking, imidoester, crosslinking, maleimide crosslinking, iodoacetamide crosslinking, benzidine crosslinking, periodate crosslinking, isothiocyanate crosslinking, and the like. Such conjugation methods may optionally use a reactive sidechain group of an amino acid residue of the binding agent (e.g., a reactive side-chain group of a Lys, Cys, Ser, Thr, Tyr, His or Arg amino acid residue of the protein, i.e., a polypeptide linking group may be amino-reactive, thiol-reactive, hydroxyl-reactive, imidazolyl-reactive or guanidinyl-reactive). In some cases, a chemoselective reactive functional group may be utilized. Other conjugation reagents that can be used include, but are not limited to, e.g., homobifunctional conjugation reagents (e.g., (bis(2-[succinimidooxycarbonyloxy]ethyl) sulfone, 1,4-Di-(3′-[2′pyridyldithio]-propionamido) butane, disuccinimidyl suberate, disuccinimidyl tartrate, sulfodisuccinimidyl tartrate, dithiobis(succinimidyl propionate), 3,3′-dithiobis(sulfosuccinimidyl propionate), ethylene glycol bis(succinimidyl succinate), and the like), heterobifunctional conjugation reagents (e.g., m-maleimidobenzoyl-N-hydroxysuccinimide ester, m-maleimidobenzoyl-N-hydroxysulfosuccinimide ester, N-γ-maleimidobutyryloxysuccinimide ester, N-γ-maleimidobutyryloxysulfosuccinimide ester, N-(8-maleimidocaproic acid) hydrazide, N-(ε-maleimidocaproyloxy) succinimide ester, N-(8-maleimidocaproyloxy) sulfo succinimide ester, N-(p-maleimidophenyl) isocyanate, N-succinimidyl (4-iodoacetyl)aminobenzoate, succinimidyl 4-(N-maleimidomethyl) cyclohexane-I-carboxylate, succinimidyl 4-(p-maleimidophenyl) butyrate, N-sulfosuccinimidyl(4-iodoacetyl)aminobenzoate, sulfosuccinimidyl 4-(N-maleimidomethyl) cyclohexane-I-carboxylate, sulfo succinimidyl 4-(p-maleimidophenyl) butyrate, I-ethyl-3-(3-dimethylaminopropyl) carbodiimide hydrochloride, I-ethyl-3-(3-dimethylaminopropyl) carbodiimide hydrochloride, maleimide PEG N-hydroxysuccinimide ester, and the like), photoreactive conjugation reagents (e.g., p-azidobenzoyl hydrazide, N-5-azido-2-nitrobenzyloxysuccinimide, p-azidophenyl glyoxal monohydrate, N-(4-[p-azidosalicylamido]butyl)-3′-(2′-pyridyldithio) propionamide, bis(P-[4-azidosalicylamido]-ethyl) disulfide, N-hydroxysuccinimideyl-4-azidosalicyclic acid, N-hydroxysulfosuccinimidyl-4-azidobenzoate, sulfosuccinimidyl 2-(7-azido-4-methylcoumarin-3-acetamide) ethyl-1,3-dithiopropionate, azido phenyl 2-(m-azido-o-nitrobenzamido)-ethyl-1,3′-propionate, sulfosuccinimidyl 6-(4′-azido-2′-nitrophenylamino) hexanoate, sulfosuccinimidyl(4-azidophenyl dithio) propionate, sulfosuccinimidyl-2-(p-azidosalicylamido)ethyl-1,3-dithiopropionate, and the like).

In some instances, attachment of a fluorophore to a binding agent of interest is mediated by one or more functional linkers. A functional linker, as used herein, refers to any suitable linker that has one or more functional groups for the attachment of one molecule to another. For example, in some instances the functional linker comprises an amino functional group, a thiol functional group, a hydroxyl functional group, an imidazolyl functional group, a guanidinyl functional group, an alkyne functional group, an azide functional group, or a strained alkyne functional group. Further exemplary functional groups and methods of crosslinking and conjugation are described in, e.g., Hermanson Bioconjugate Techniques (Academic Press, 3rd edition, 2013), herein incorporated by reference in its entirety.

Once a dye or probe is applied to a particular tissue, it can be imaged with an imaging chip as described herein. Such imaging can comprise bringing a composite filter in proximity with tissue. Proximity can include contacting tissue with a composite chip or bringing them to within a small distance to each other. Such a small distance can range from less than 1 mm up to approximately 10 mm, such as approximately 0.1 mm, 0.25 mm, 0.5 mm, 0.75 mm, 1 mm, 2 mm, 3 mm, 4 mm, 5 mm, 6 mm, 7 mm, 8 mm, 9 mm, or 10 mm.

Once in proximity to a tissue, the tissue can be imaged. Imaging can include illuminating tissue with white light and/or a subset of light (e.g., specific wavelengths and/or ranges of wavelengths). For example, illumination can occur with light that overlaps with a fluorophore's excitation spectrum or excitation maximum. Imaging can utilize a single exposure or multiple exposures which can be combined or averaged. Single exposure imaging can be of any amount of time to allow for sufficient imaging of the probe or dye. Exposure times can be from approximately 10 milliseconds (ms) up to approximately 1 second, such as 10 ms, 25 ms, 50 ms, 75 ms, 100 ms, 150 ms, 200 ms, 250 ms, 500 ms, 750 ms, or 1000 ms (1 second). When taking multiple exposures, any number of exposures may be used with any exposure length. For example, 5 to 250 exposures may be used, such as 10 exposures, 25 exposures, 50 exposures, or 75 exposures, 100 exposures, 150 exposures, 200 exposures, or 250 exposures.

Imaging can include multichannel imaging, such as when using multiple probes and/or dyes, where each probe or dye possesses a different excitation and/or emission wavelength. In multichannel imaging, each channel can be imaged consecutively (e.g., one at a time) or simultaneously. However, simultaneous imaging may require a polychromatic imaging sensor. Additionally, multichannel imaging can use a different exposure time and/or number of exposures for each channel.

Further embodiments can further resect imaged tissue to remove cancerous or diseased tissue. Cancerous or diseased tissue can be identified based on the fluorescent dyes and/or probes that are used and described above. In certain instances, probes or dyes mark healthy tissues, so non-marked tissue is excised. In other instances, probes or dyes mark diseased tissues, so marked tissue is excised. Additional instances use a first dye that is specific for healthy tissue and a second dye that is specific for diseased tissue; in these situations, the tissue marked as diseased is removed.

In ex vivo imaging, tissue samples (such as described above) can be imaged by a composite filter and imaging sensor. Such imaging can reveal presence or absence of certain targets, such as target cells or target tissues. Such targets can include diseased tissues, such as cancerous, neoplastic, infected, and/or any other form of diseased tissue.

In addition to or instead of ex vivo imaging, certain embodiments perform in vivo imaging. During in vivo imaging, diseased tissue can be resected from the healthy tissue, based on the tissue identified by imaging. Resection can be simultaneous with imaging or following imaging, such that resection can occur during the imaging process or resection can occur once the boundaries or margins between diseased tissue and health tissue are identified.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

### Computer Readable Media and Devices

Also provided by the present disclosure are computer-readable media and devices. For example, one or more steps of any of the methods of the present disclosure may be computer-implemented. By “computer-implemented” generally means at least one step of the method is implemented using one or more processors and one or more non-transitory computer-readable media. The computer-implemented methods of the present disclosure may further comprise one or more steps that are not computer-implemented.

As will be appreciated with the benefit of the present disclosure, any of the methods of the present disclosure amenable to computer-implementation may be implemented in a similar manner employing one or more processors and one or more non-transitory computer-readable media comprising instructions stored thereon, which when executed by the one or more processors, cause the one or more processors to perform one or more steps of such methods.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

The following examples provide details on certain embodiments. These examples are exemplary and/or illustrative in nature and are not intended to limit the scope of the invention.

### EXPERIMENTAL

### Example 1: Collimator and Interference Filter Configuration

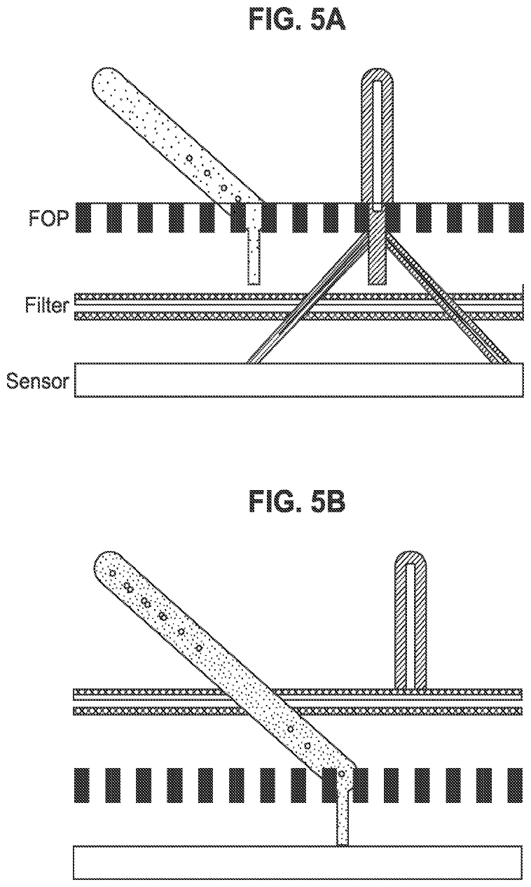

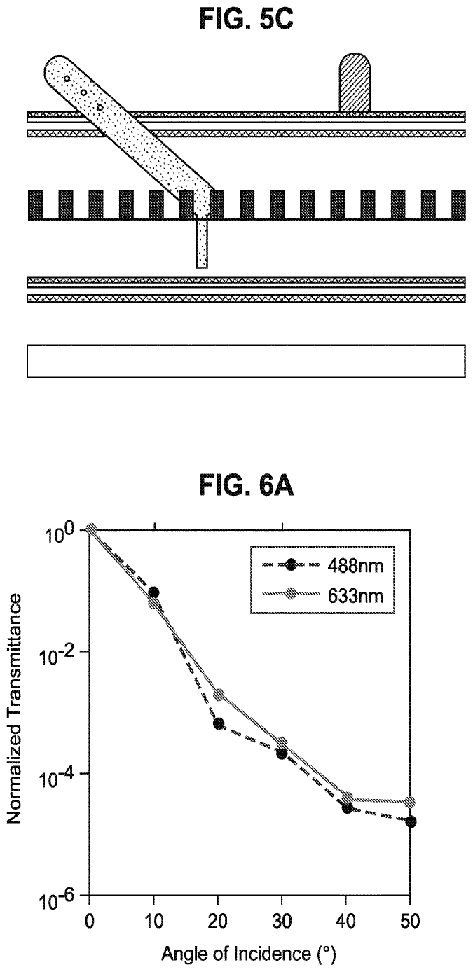

The order in which the fiber optic plate (FOP) and interference filter are stacked on the imager has an effect on filter performance depending on whether oblique or near-normal incident excitation is used (FIGS. 5A-5C). This dependence is due to the combination of the angular sensitivity of the interference filter and scattering from the FOP. Scattering in the FOP is caused both by material imperfections and by diffraction effects due to the micron-scale fiber apertures.

First, consider the case when excitation is near normal incidence. In this case, the excitation light incident on the FOP (this can also be a more generalized collimator plate, with any of the compositions listed above) passes through the fibers. As the light exits the fibers, a small fraction of the light is scattered to larger angles. Consequently, if the FOP is placed on top of the filter (FIG. 5A), excitation light scattered at large AOIs will transmit directly through the filter as described in the preceding section. This effect can be minimized by placing the interference filter on top of the FOP (FIG. 5B). Since the interference filter provides strong excitation rejection near normal incidence, an insignificant amount of excitation light will reach the sensor. Therefore, for excitation near normal incidence the interference filter is responsible for providing excitation rejection and should preferentially be placed on top of the FOP. However, there may be situations in which the FOP (or, in general, the parallel-hole collimator) should be placed on the interference filter.

An alternative case is when the excitation light is incident with a large AOI. When excitation light is incident on the FOP with AOIs larger than the NA of the fibers, the light will pass through the EMA sidewalls and experience significant attenuation. However, a small fraction of the incident light scatters through the apertures, escaping the absorptive side walls, and exiting the FOP at near-normal incidence to the sensor. Consequently, it is preferred to place the interference filter below the FOP to block the excitation that is scattered. If the interference filter is placed above the FOP, the excitation rejection will be limited by the scattering effects and not the absorption performance of the FOP. Thus, for obliquely incident excitation the FOP is responsible for providing excitation rejection and should be placed on top of the interference filter. Throughout this disclosure, the mention of an FOP is also interchangeable with parallel-hole collimator, as different compositions of the device may achieve the desired overall function.

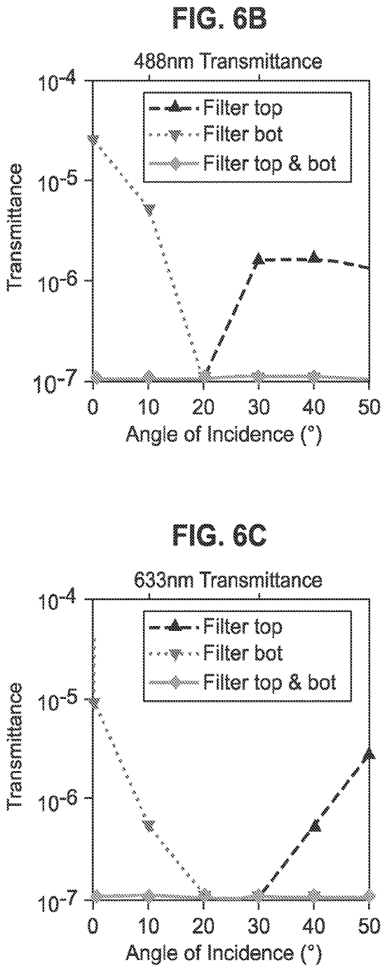

Measurements of the angular transmittance of the FOP at 488 nm and 633 nm (FIG. 6A), show that the transmittance of the FOP starts to plateau around 40° at 5 orders of magnitude rejection due to scattering effects. To include scattered light in the measurement, the FOP is placed in direct contact with the photometer such that all transmitted light is captured. Similar measurements are performed for both orientations of the optical front-end (FIGS. 6B-6C). The optical front-end best blocks excitation near normal incidence when the interference filter is on top and shows the highest performance for oblique excitation with the FOP on top. The difference in performance between the two orientations at the extremes of both regimes is between 1-2 orders of magnitude. The measurements also show that the angle-sensitivity of the front-end can be completely rectified by placing the same interference on both sides of the FOP. With this modification the filter shows superior performance for both normal incident and oblique excitation. This design comes at the cost of added fabrication complexity and is only necessary when the AOI of the excitation light is not known. As discussed earlier, for contact imaging applications, the excitation may be introduced obliquely, so it is sufficient to have a single interference filter on the bottom of the FOP.

### Example 2: Characterizing Optical Front Ends

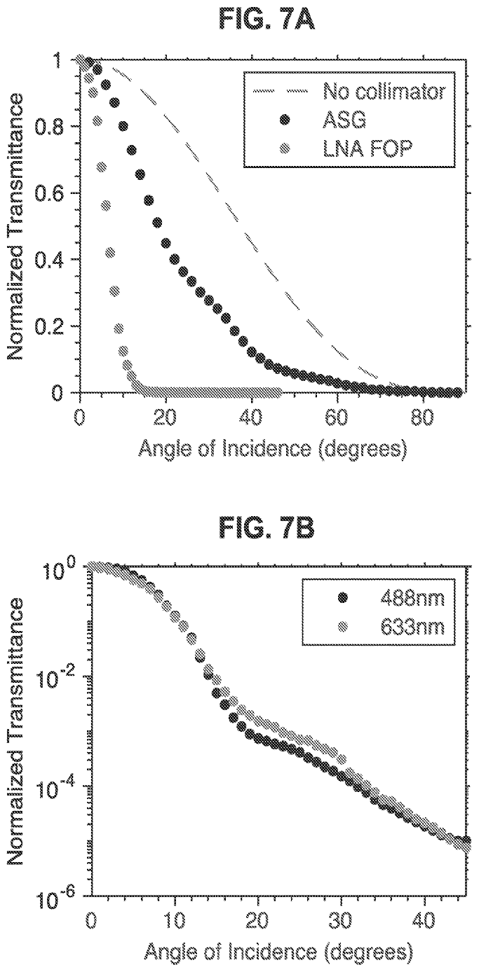

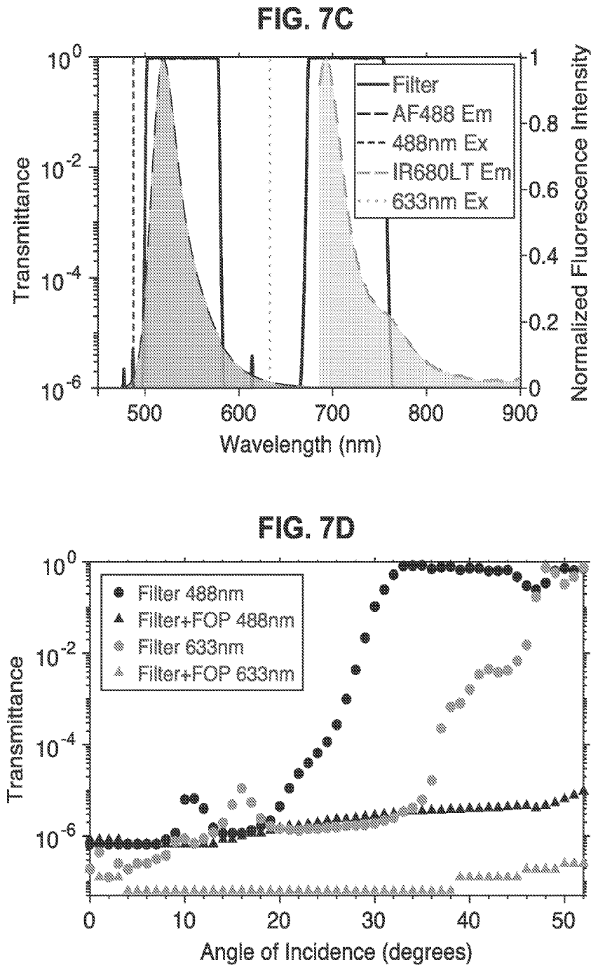

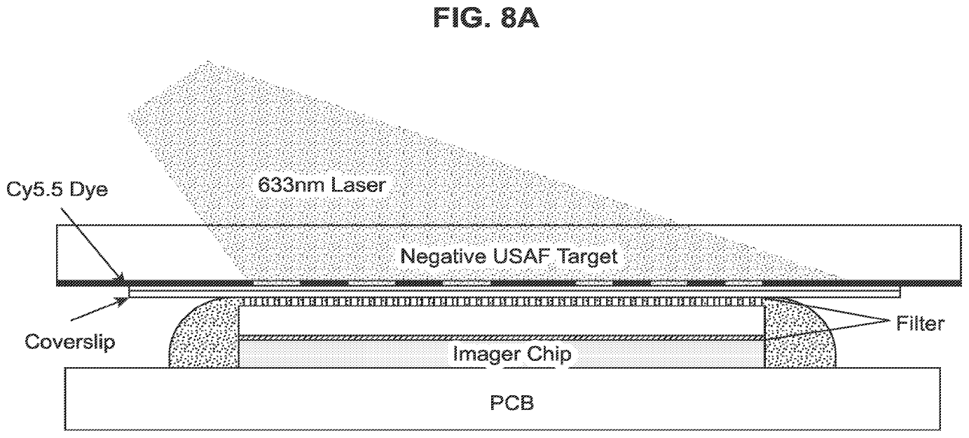

FIGS. 7A-7D are graphs illustrating example characterizations of an optical front-end designed in accordance with the techniques described herein. In FIG. 7A, a comparison of angular selectivity of FOP, on-chip collimators made of an angle-selective grating (ASG), and a lens-less imager with no collimators is shown. The FOP compared to the ASGs reduces the FWHM by almost 3× to 12.65°. To validate the design, the performance of the FOP, interference filter, and final filter design was measured and characterized at different AOIs on an optical breadboard setup. FIG. 7A shows the measured angular transmittance of the FOP in air at 488 nm compared to the on-chip angle-selective gratings (ASGs) and the theoretical angular sensitivity of a lens-less imager without collimators. The full width at half maximum (FWHM) of the FOP—which is indicative of the resolution of the imager—was found to be 12.65°, almost 3× smaller than that of the ASGs (36°) and significantly smaller than that of the lens-less imager without collimators (75°). This improvement over ASGs is due to the fact that on-chip collimators may be constructed with reflective metal as opposed to optically absorbing material and have aspect ratios limited by the thickness of on-chip metal layers and design rules of the process. The fill factor of the EMA glass also limits the normal transmittance of the FOP, which was found to be 40%.

In FIG. 7B, an angular transmittance of FOP on a log scale is shown. The FOP achieves more than five orders of magnitude rejection of both excitation wavelengths by 45°. To demonstrate the absorptive properties of the FOP necessary for interference filter compensation, FIG. 7B shows the same angular transmittance measurement of the FOP on a log scale for excitation wavelengths of 488 nm and 633 nm. Here, it is shown that at an AOI of 45° the FOP already achieves 5 orders of magnitude rejection for both wavelengths.

In FIG. 7C, a spectra of multi-bandpass interference filter are shown. The filter provides more than 6 orders of rejection of 488 nm and 633 nm excitation while passing a majority of emitted fluorescence for various filters. The spectral design for the imager, including the transmittance spectra of the interference filter and the emission spectra for two particular fluorophores (AlexaFluor488 and IRDye680LT), with corresponding excitation laser lines at 488 nm and 633 nm—is shown in FIG. 7C. The interference filter provides sufficient bandwidth to capture emitted fluorescence while providing 6 orders of magnitude of excitation rejection.

In FIG. 7D, an angular transmittance of interference filter with and without FOP at both excitation wavelengths is shown. Without the FOP, the filter becomes practically transparent to 488 nm at 30° and 633 nm at 45°. The FOP effectively compensates for this behavior, maintaining more than 5 orders of magnitude rejection at 488 nm and 6 orders at 633 nm. As a proof of concept of the filter design, the angular transmittance of the interference filter was measured with and without the FOP at both excitation wavelengths (FIG. 7D). For the interference filter, at small AOIs the filter maintains adequate excitation rejection, but near 20° for 488 nm and 35° for 633 nm, the transmittance sharply increases as the filter passband shifts over the excitation wavelengths, becoming practically transparent at 33° and 48°, respectively. The 488 nm excitation shifts into the filter passband at a smaller AOI as it is closer to the filter band-edge than 633 nm. When the FOP is added to compensate the interference filter, the composite filter maintains more than 5 orders of magnitude rejection at 488 nm and more than 6 orders of magnitude rejection at 633 nm. Although the rejection from the interference filter degrades at higher AOIs, the FOP displays the complementary behavior, becoming increasingly absorptive and preserving adequate excitation rejection for fluorescence imaging.

### Example 3: Imaging Resolution

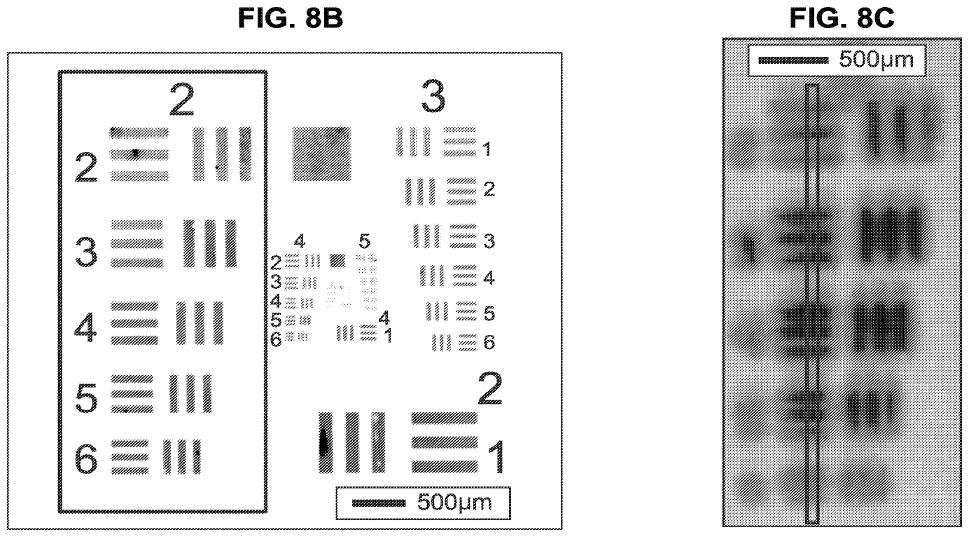

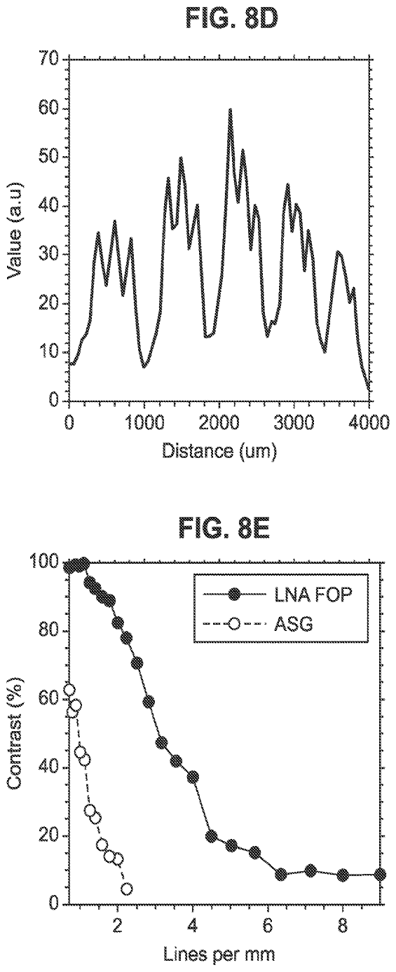

FIGS. 8A-8E are example resolution measurements of an optical front-end designed in accordance with the techniques described herein. In FIG. 8A, an experimental setup for resolution measurements with USAF 1951 test target is shown. For fluorescence imaging, the test target is used to pattern excitation light onto a uniform layer of Cy5.5 dye contained with a coverslip on the surface of the target. The target assembly is placed directly on an imaging chip described herein. In order to determine the resolution of the imager with the FOP, the system imaged a standard negative United States Air Force 1951 (USAF-1951) resolution test target. For fluorescence imaging, the USAF target is used to pattern excitation light onto a uniform layer of Cy5.5 dye, which is placed directly on the imaging chip (FIG. 8A). In FIG. 8B, a reference image of test target taken at 2.5× using a benchtop fluorescence microscope is shown.

In FIG. 8C, an image taken with the imaging chip of group 2 on the test target is shown, which lies near the resolution limit of the sensor. The reference image of FIG. 8B can be compared with the chip image of the elements in group 2 (FIG. 8C), which lay at the resolution limit of the sensor.

In FIG. 8D, a line scan through all horizontal elements in the image in FIG. 8C. As the line spacing approaches, the resolution limit, the contrast decreases. For a more quantitative analysis of the imager resolution, the contrast transfer function (CTF) of the imager was measured. This was achieved by taking individual images of each three-bar element in the USAF target and plotting the contrast of each element as measured from a line scan of the image (FIG. 8D) versus the spatial frequency of the element.

In FIG. 8E, a measured contrast transfer function of imager with Low-NA FOP vs. on-chip collimators (ASGs) is shown. With LNA FOP, the imaging chip achieves 3× resolution improvement at 20% contrast compared with previous work. To compare the resolving power of the FOP vs. the ASGs, the CTFs of both an imager chip with the proposed filter design and previous imagers were measured (FIG. 8E). These measurements show that with the FOP, the imager can resolve spatial frequencies of 4.5 lpmm (line widths of 110 μm) with 20% contrast, while with the ASGs, the imager is only capable of resolving spatial frequencies of 1.5 lpmm (line widths of 330 μm) with 20% contrast, representing a 3-fold improvement in resolution. The achieved resolution is near the pixel size of the imager (55 μm).

### Example 4: Tumor Resection

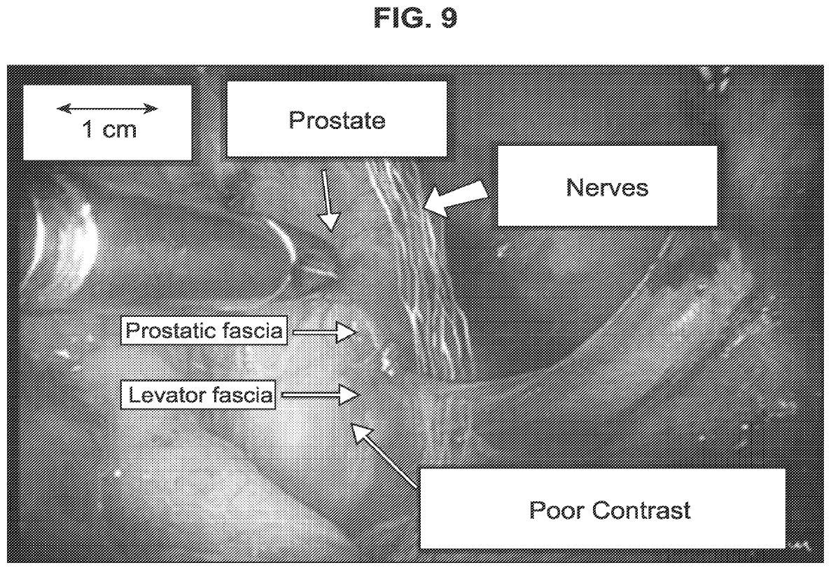

FIG. 9 is an image illustrating various fascia on and near a prostate. A goal in tumor resection surgeries is the complete removal of all gross and microscopic disease with minimal damage to neighboring healthy tissue. However, in the operating room, surgeries are primarily guided through visual examination, touch, or white light imaging. Because these techniques lack adequate contrast, many surgeries fall short of this goal. For example, positive margins, where cancer cells are detected along the margin of the tumor cavity indicating an incomplete resection, are common across many cancer types. In breast cancer operations, the rate of occurrence is estimated to be as large as a quarter of all patients. This number is greater than 20% for prostate cancer patients. Overall, what this means, is that effected patients have a two-times increased risk of recurrence. There are also additional problems related to this imaging gap. Nodal disease can often go undetected, increasing the risk of metastases. Unseen healthy structures, such as nerves, can be damaged during the surgery, often leading to lifelong complications for the patient.

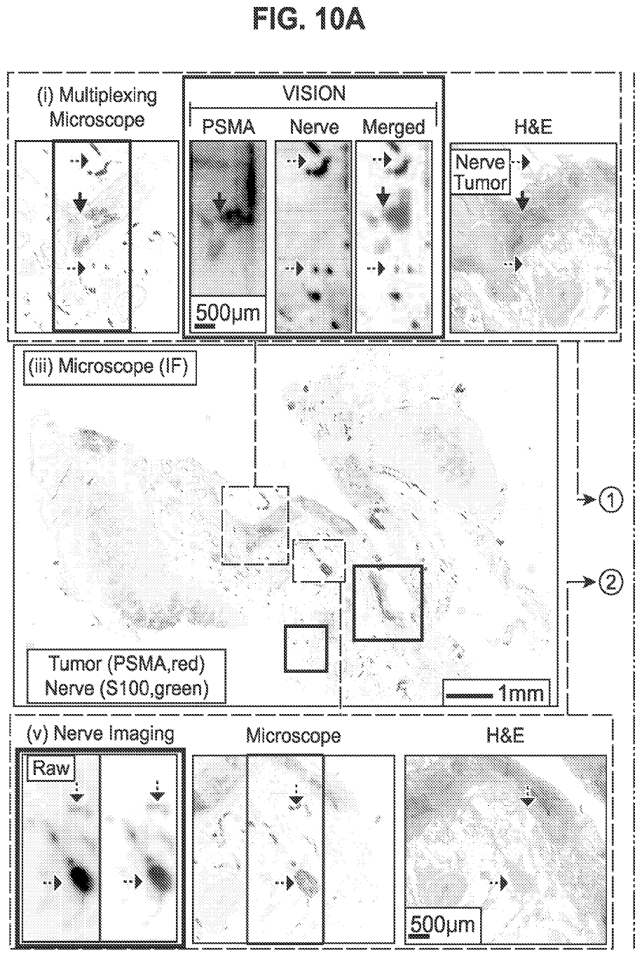

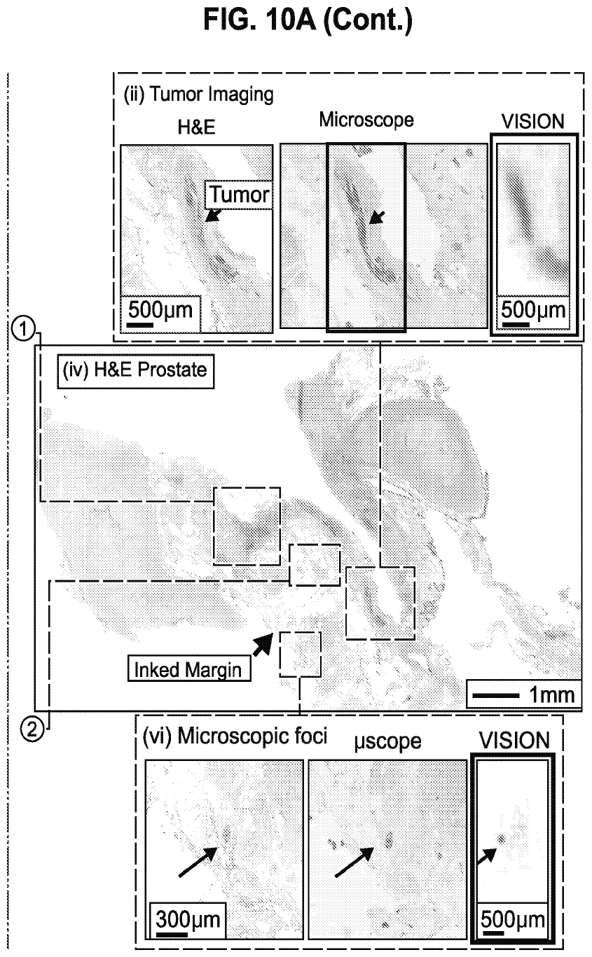

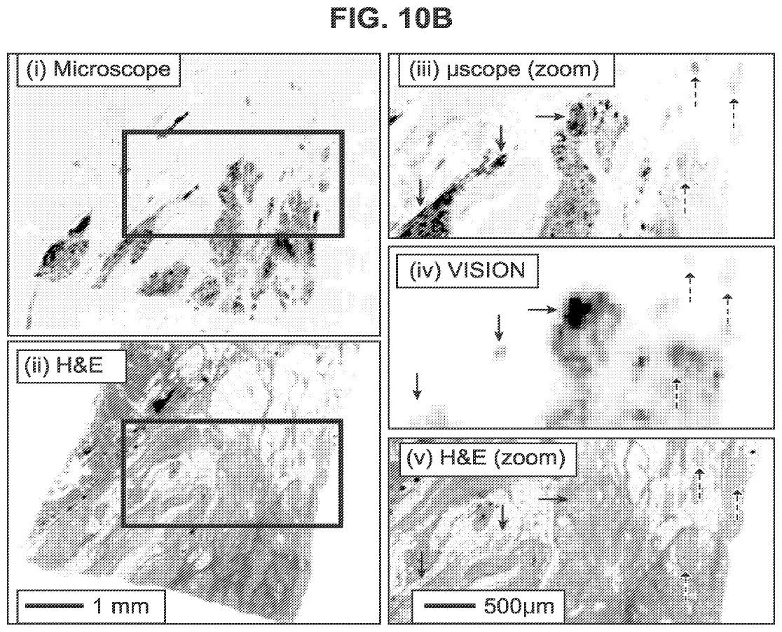

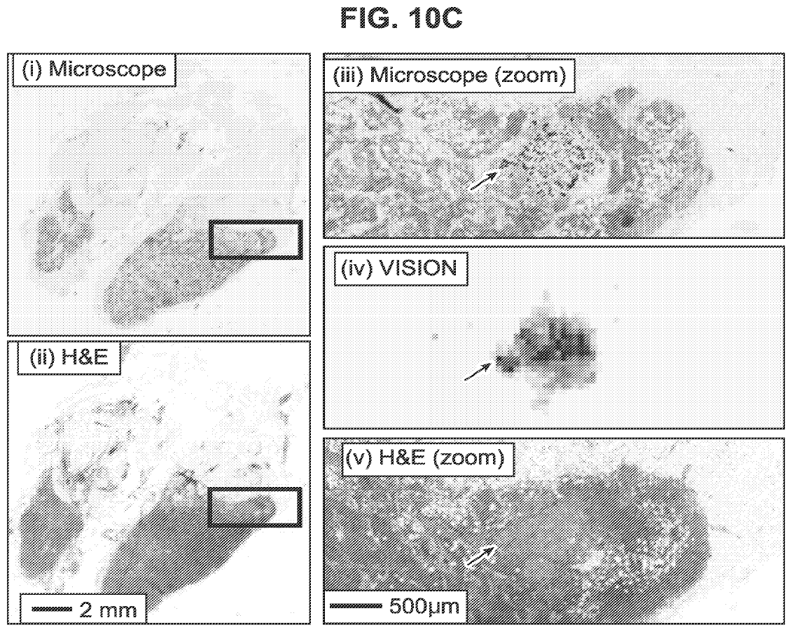

FIGS. 10A-10C are examples of ex vivo imaging of resected prostate tissue using an optical front-end designed in accordance with the techniques described herein. To demonstrate (ex vivo) imaging of clinically relevant scenarios in prostate cancer with an imaging chip prototype (notated as VISION), the system imaged banked, resected patient tissue with tumor and nerves. The tissue samples are paraffin embedded, sectioned at 4 μm thickness, and mounted onto glass slides. The samples were fluorescently stained for both prostate cancer with an anti-PSMA antibody conjugated to red and nerves with an anti-S100 antibody conjugated to green. To verify staining, slides are scanned with a fluorescence microscope and compared against full-slide H&E scans by a trained pathologist. Relevant areas of each slide are then imaged with VISION. Dual-color imaging of both fluorescence channels is achieved with a single chip by taking a separate exposure with each excitation wavelength. To improve image quality, multiple exposures are captured and then averaged for each channel. For the prostate channel, 100 exposures at 75 ms are used for each image, while for nerves, 100 exposures at 50 ms are used.

In FIG. 10A, images of resected prostate tumor immuno-fluorescently (IF) stained for prostate cancer with red and nerves green are shown. Comparison with full slide microscope scans of IF (iv) and H&E staining (iv) shows that VISION can clearly identify tumor (ii) and nerves (v). Simultaneous detection of both tumors and nerves with the same imager (i) is possible by overlaying separate exposures of each channel. (vi) VISION is highly sensitive and is able to detect a microscopic foci (<100 cells) along the inked margin. FIG. 10A shows a side-by-side comparison of images of a resected prostate tumor captured with VISION and a benchtop fluorescence microscope alongside H&E images. VISION clearly identifies tumor foci and nerves, including a microscopic tumor foci of less than 100 cells along the inked margin of sample.

In FIG. 10B, images of extra-prostatic extension (EPE) into fibroadipose tissue are shown. VISION (iv) is able to clearly identify most tumor and nerves identified in microscope IF (i, iii) and histological (ii, v) references. In FIG. 10C, images of metastatic tumor foci in lymph node are shown. VISION detects sight of metastatic tumor (iv) visible in microscope IF (i, iii) and histological (ii, v) references. In FIGS. 10B-10C, two other clinically relevant examples were imaged using VISION to visualize extra-prostatic extension (EPE) of tumor into fibroadipose tissue interspersed with nerves and to identify a metastatic lymph node. Overall, these images illustrate the potential use of VISION to intraoperatively assess resection margins for microscopic residual disease, provide critical information regarding the spatial proximity of tumor and nerves for surgical guidance, and identify sites of metastatic spread.

### Example 5: Imaging Chip Construction

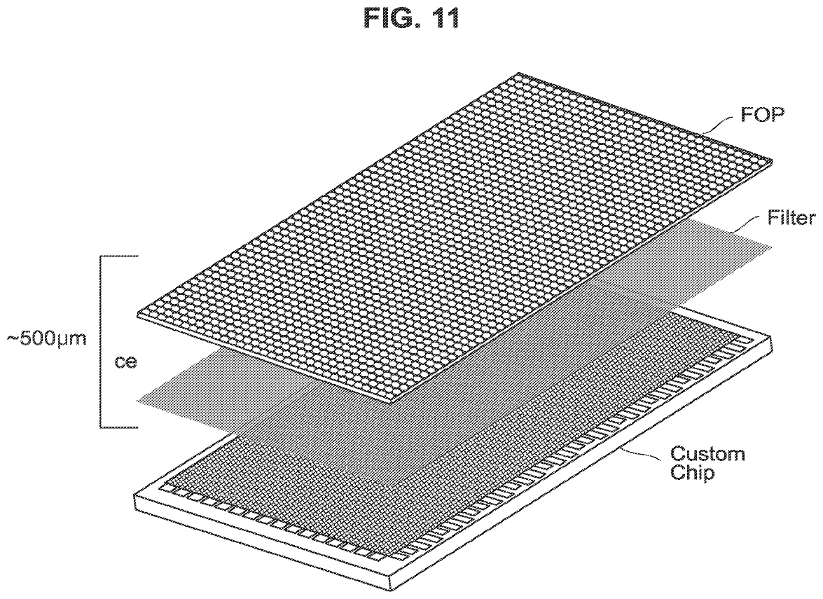

FIG. 11 is a conceptual diagram of an example interference filter directly coated on a fiber optic plate (FOP), in accordance with the techniques described herein. The techniques described herein fabricate the optical front-end by directly coating a multi-bandpass interference filter on top of a Low-NA fiber optic plate. The filter has two bands: one in the green region and another in the NIR band, with good spectral characteristics which excite at 488 nm and 633 nm. The interference filter shows significant excitation bleed through at large angles of incidence. However, the FOP is highly absorptive at large angles of incidence, compensating for this deficiency.

### Example 6: Cell Culture Imaging

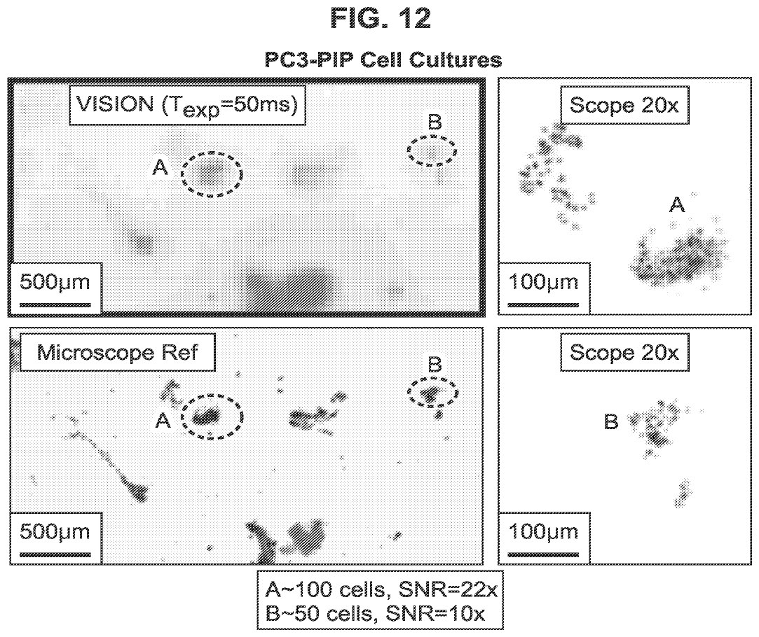

FIG. 12 is an example of imaging of PC3-PIP cell cultures, in accordance with the techniques described herein. A system as described herein imaged PC3-PIP cell cultures stained with anti-PSMA with dyes in both channels. The system imaged FoVs containing cell clusters of different sizes with a single 50 ms exposure time and measured the highest at pixel signal to noise ratio. In the image on the left the two clusters labeled A and B contain cell clusters of 100 and 50 cells and are imaged with SNRS of 22 and 10, respectively.

### Example 7: Signal Noise Ratios

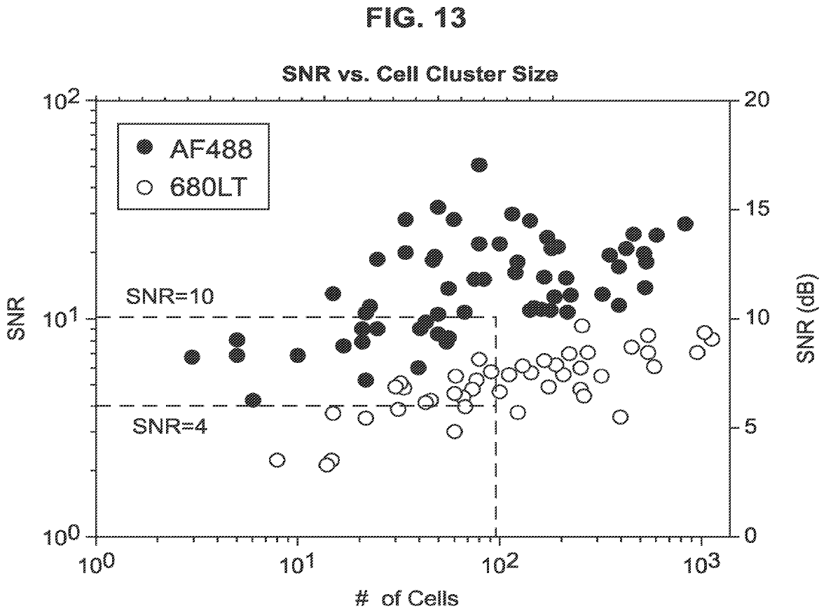

FIG. 13 is a graph illustrating signal noise ratios vs. cell cluster sizes, in accordance with the techniques described herein. FIG. 13 shows that a system described herein is capable of imaging clusters as small as 100 cells with an SNR of 10 dB, meaning that these signals are more than 10× above our noise level. And the NIR channel 100 cell clusters are visible at SNRs of 6 dB. The SNR can be significantly improved using longer exposure times, higher laser powers, and the use of better CMOS technologies, which can move toward single cell detection.

### Example 8: Signal Noise Ratios

An imaging system is typically characterized by its point spread function (PSF). The PSF is equivalent to the impulse response function of a linear shift invariant (LSI) system and can be determined by finding the image produced by the system when imaging an ideal point source. Any LSI system can be modeled as convolution between the PSF and the sample plane S(x, y):

I(x,y)=S(x,y)*PSF(x,y) (1)

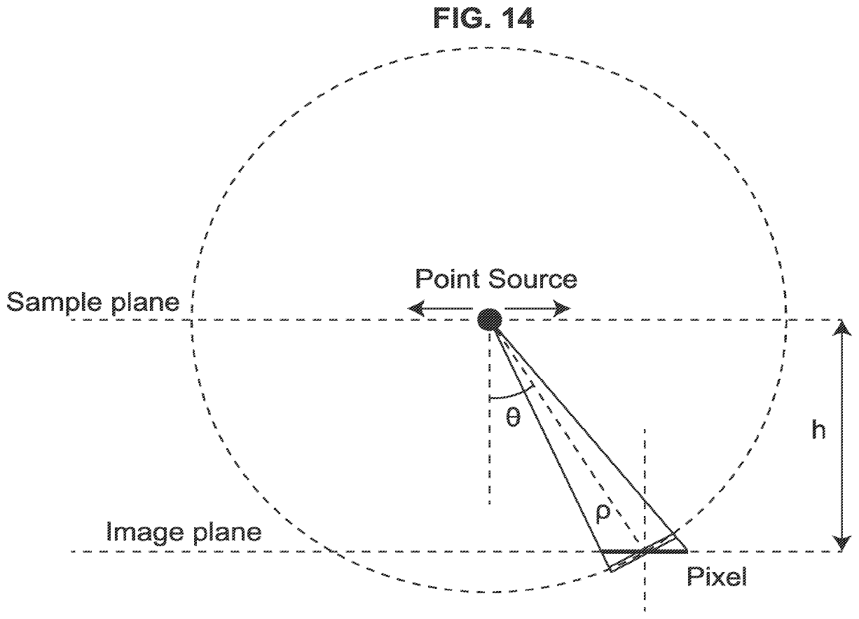

In this way, the PSF acts as a blurring kernel, spreading out each point source in the sample plane. This can be quantified by measuring full width at half maximum (FWHM) of the PSF. The following methods include references to angles, lengths, and other aspects of geometry, which are illustrated in FIG. 14. In systems with symmetry around the z-axis, it is often convenient to reduce to PSF to a single dimension, PSF(θ)=PSF(x, y), where θ is the angle between z axis and the vector from origin of the sample plane to a point in the image plane such that

$$tan ( θ ) = \frac{\sqrt{{x}^{2} + {y}^{2}}}{h} ,$$

where h is the vertical separation between the imager and the sample. In the case that PSF(θ) does not vary with depth,

$${FWHM}_{x , y} = 2 h \cdot tan ( \frac{{FWHM}_{θ}}{2} ) . \qquad ( 2 )$$

The approximate PSF of a general lens-less imaging system can be derived as follows. To simplify to a single dimension, we assume a small circular pixel. Then, PSF(θ) is found by placing a point source on the sample plane located at angle θ off the z axis from the pixel at origin on the imager plane and determining fraction of total light emitted by the point source that is incident on the pixel for each θ,

$$PSF ( θ ) ∝ \frac{light incident on pixel ( θ )}{total emitted light} \qquad ( 3 )$$

The sample plane lies at vertical distance h from the imager, such that the total distance from the point source to the pixel is

$$ρ = \frac{h}{cos ( θ )} . \qquad ( 4 )$$

The point source emits isotopically with its total surface flux, Φ, at radial distance p inversely proportional the area of the sphere defined by ρ,

$$Φ ∝ \frac{1}{4 {πρ}^{2}} \qquad ( 5 )$$

The pixel subtends a cone of this sphere defined by the perimeter of the pixel. Thus, the effective area of the pixel from the perspective of the point source can be found as the area of the elliptical pixel at the base of this cone that is tangent to the sphere,

A_{pixel eff}=Aₚᵢₓₑₗ·cos(θ) (6)

In lens-less imaging, the pixel pitch (~5-55 μm) is often much smaller than the separation distance (0.5-1.5 mm) due to the filter thickness and spacer necessary for epi-illumination of the sample. As a result, we can assume that the area of the spherical patch at the base of the cone is equivalent to the effective area of the pixel. Therefore, replacing equations (4), (5), and (6) into (3) and removing all constants from the proportionality, it is found:

PSF(θ)∝Φ·A_{pixel eff}∝ cos³(θ) (7)

The cubic dependency on θ comes from the fact that as the point source moves further from the pixel the intensity degrades quadratically and the effective area of the pixel shrinks linearly with distance. This PSF has a FWHM_{θ} of 75° and, following (2), the FWHM_{x,y} will increase linearly with the separation distance. As a result, lens-less imagers naturally have very poor lateral resolution. For example, at 1 mm separation distance, a FWHM of 1.5 mm is expected.

To be sure, the above model only considers a single 2D sample plane. For generalized 3D imaging, the image of each 2D slice is super-imposed on the image plane. In addition, scattering and absorption effects may be taken into account for each layer requiring a more complex model. In general, the imaging depth will be limited to a couple of mm as there is significant attenuation with increasing depth due to the absorption and scattering of tissue, which are high for visible and NIR wavelengths, and quadratic spreading loss of the fluorescence emissions.

To improve imager resolution, a collimator can be used to further restrict the pixel angle of view (AoV). Typical lens-less collimators are Parallel-hole collimators, which are composed of an array of holes within an absorptive media. Light at AOIs close to normal incidence see a direct path through the collimator, while at oblique AOIs the light may pass through the sidewalls of the collimator and is attenuated. The angle-selectivity of a Parallel-hole collimator is determined directly by the aspect ratio (channel length/hole diameter) of the holes. Larger aspect ratios provide sharper angle selectivity.

The relation derived above for the PSF of a lens-less imager can be modified to incorporate the effect of additional angle selectivity provided by the collimator. Under the assumption that the pixel is very small relative to the separation distance, a pixel on the imager located at θ from the point source sees the incident intensity modulated by the angular transmittance of the collimator f(θ) at θ. As such the PSF becomes,

PSF(θ)∝f(θ)·cos³(θ) (8)

This relation shows that for collimators with an angle selectivity significantly stronger that the natural PSF of the imager, the PSF and, therefore, the resolution, is determined almost entirely by the angular transmittance and FWHM of the collimator. Therefore, better resolution can be achieved simply by choosing a collimator with a higher aspect ratio and smaller FWHM.

Of course, the increased angle selectivity afforded by the collimator comes at the cost of reduced fluorescence signal seen by the sensor. Signal is lost both due to the fill factor of the collimator (percentage of collimator surface that is transparent at normal incidence) and the fact that obliquely incident light is absorbed. In fluorescence imaging applications, where the photon budget is inherently limited due to small absorption cross-sections of the dyes, this trade-off is of particular concern.

Generally, the efficiency of an imaging system in collecting available light from the sample is quantified by the collection efficiency, the percentage of total emitted light from a point source that is collected by the system. For a general system, the collection efficiency, η_{collect}, is found as the fractional surface area of a sphere surrounding the point source that is covered by the imager taking into account loss due to the angular transmittance, f(θ), of the system,

$${η}_{collect} = \frac{1}{2} {\int }_{0}^{m ax} f ( θ ) \cdot sin ( θ ) d θ , \qquad ( 9 )$$

where θₘₐₓ is the angle between the vector connecting the perimeter of the imaging system and the point source and normal vector of the imaging plane assuming a circular imaging geometry. The maximum collection efficiency is 50% due to the fact that a planar sensor with infinite area only collects light from one side of the point source. To understand the relationship between collection efficiency and the angle selectivity of an imaging system, it is useful to look at the ideal case in which the imager has a ‘brick-wall’ angular transmittance defined by a cut off angle θ_{C}. In this case,

$$f ( θ ) = \{ \begin{matrix}1 , θ < {θ}_{c} \\ \frac{1}{2} , θ = {θ}_{c} \\ 0 , θ > {θ}_{c}\end{matrix} \qquad ( 10 )$$

Solving (8), it is found

$${η}_{collect} = {sin}^{2} \frac{{θ}_{c}}{2} . \qquad ( 11 )$$

Thus, for small θ_{c}, the collection efficiency has a roughly quadratic dependence on θ_{c}, whereas the FWHM has an approximately linear proportionality to θ_{c}. The insight here is that improvements in resolution by reducing pixel AoV through the use of collimators come at costs in total signal loss. This tradeoff represents the primary disadvantage of lens-less imaging using collimators compared with lensed approaches. In conventional cameras and microscopes, the lens forms an image by focusing divergent rays to back to a point. As a result, there is no tradeoff between resolution and collection efficiency and both are improved with increased aperture size.

In in vivo fluorescence imaging, detection is determined by both the signal to noise ratio (SNR) The background signal received by the sensor comes from the combination of ambient light, excitation light bleed-through, free-floating dye, and/or non-specific binding of dyes or probes.

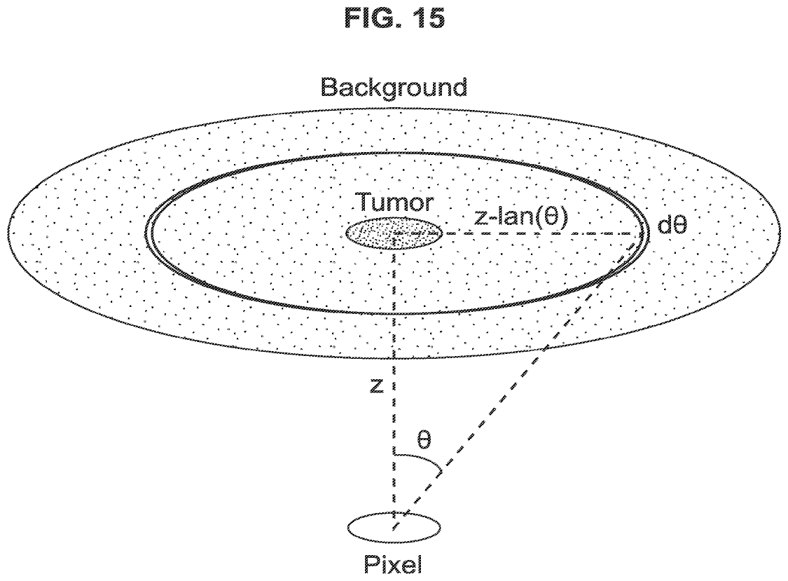

In the most extreme case, a small, fluorescently labeled, tumor foci are completely surrounded by healthy tissue that contributes a fluorescent background signal. We model this situation by considering a 2D plane with z-axis symmetry where small circular region of tumor is embedded within an infinite plane of background (FIG. 15). From the geometry of the model and by assuming a small pixel relative to the separation distance, z,

$$S = {{\int }_{0}}^{\frac{π}{2}} tan ( θ ) \cdot {PSF}_{point source} ( θ ) d θ \\ T B R = \frac{P\_tumor}{P\_background}$$

Interference filters consist of alternating layers of materials with different refractive indices. By finely controlling the thickness of each layer such that specific wavelengths of light either constructively or destructively interfere at each interface, the filter can be designed to provide near-total transmittance of one spectral band with strong rejection of adjacent bands. However, as the angle of incidence (AOI) increases, the optical path length difference between each layer changes (FIG. 2B), altering the spectral interference of the filter and causing the overall filter response to shift to shorter wavelengths. Using simple geometric optics (FIG. 2B), it can be shown that for small AOIs the peak transmitted wavelength of a general interference filter shifts according to

$${λ}_{new} = {{λ}_{peak} [ 1 - \frac{{n}_{1}}{{n}_{2}} {sin}^{2} ( {θ}_{1} ) ]}^{1 / 2}$$

where θ₁ is the AOI and λₚₑₐₖ and

$$\frac{{n}_{1}}{{n}_{2}}$$

are the peak transmitted wavelength at normal incidence and the effective index of refraction of the filter, respectively 2. This relation shows that for small AOIs the filter response shifts negligibly, maintaining excitation rejection, while for larger AOIs, the shift is magnified, leading to increased excitation bleed-through. Therefore, even for the 488 nm laser located just 10 nm from band-edge of our filter, the FOP only needs to start to compensate for excitation bleed through at around 20°, allowing for a large enough FWHM FOP to achieve adequate collection efficiency of fluorescence emissions.

The order in which the fiber optic plate (FOP) and interference filter are stacked on the imager has a significant effect on filter performance depending on whether oblique or near-normal incident excitation is used (FIGS. 5A-5C). This dependence is due to the combination of the angular sensitivity of the interference filter and scattering from the FOP. Scattering in the FOP is caused both by material imperfections and by diffraction effects due to the micron-scale fiber apertures.

First, consider the case when excitation is near normal incidence. In this case, the excitation light incident on the FOP passes through the fibers. As the light exits the fibers, a small fraction of the light is scattered to larger angles. Consequently, if the FOP is placed on top of the filter (FIG. 5A), excitation light scattered at large AOIs will transmit directly through the filter as described in the preceding section. This effect can be reduced by placing the interference filter on top of the FOP (FIG. 5B). Since the interference filter provides strong excitation rejection near normal incidence, an insignificant amount of excitation light will reach the sensor. Therefore, for excitation near normal incidence the interference filter is responsible for providing excitation rejection and should always be placed on top of the FOP.

Now consider the case when the excitation light is incident with a large AOI. When excitation light is incident on the FOP with AOIs larger than the NA of the fibers, the light will pass through the EMA sidewalls and experience significant attenuation. However, a small fraction of the incident light scatters through the apertures, escaping the absorptive side walls, and exiting the FOP at near-normal incidence to the sensor. Consequently, the interference filter may be placed below the FOP to block the excitation that is scattered. If the interference filter is placed above the FOP, the excitation rejection will be limited by the scattering effects and not the absorption performance of the FOP. Thus, for obliquely incident excitation the FOP is responsible for providing excitation rejection.

Measurements of the angular transmittance of the FOP at 488 nm and 633 nm (FIG. 6A), show that the transmittance of the FOP starts to plateau around 40° at 5 orders of magnitude rejection due to scattering effects. To include scattered light in the measurement, the FOP is placed in direct contact with the photometer such that all transmitted light is captured. Similar measurements are performed for both orientations of the optical front-end (FIGS. 6B-6C). The optical front-end best blocks excitation near normal incidence when the interference filter is on top and shows the highest performance for oblique excitation with the FOP on top. The difference in performance between the two orientations at the extremes of both regimes is between 1-2 orders of magnitude. The measurements also show that the angle-sensitivity of the front-end can be completely rectified by placing the same interference on both sides of the FOP. With this modification the filter shows superior performance for both normal incident and oblique excitation. This design comes at the cost of added fabrication complexity and is only necessary when the AOI of the excitation light is not known. For contact imaging applications, the excitation may be introduced obliquely, so it is sufficient to have a single interference filter on the bottom of the FOP.

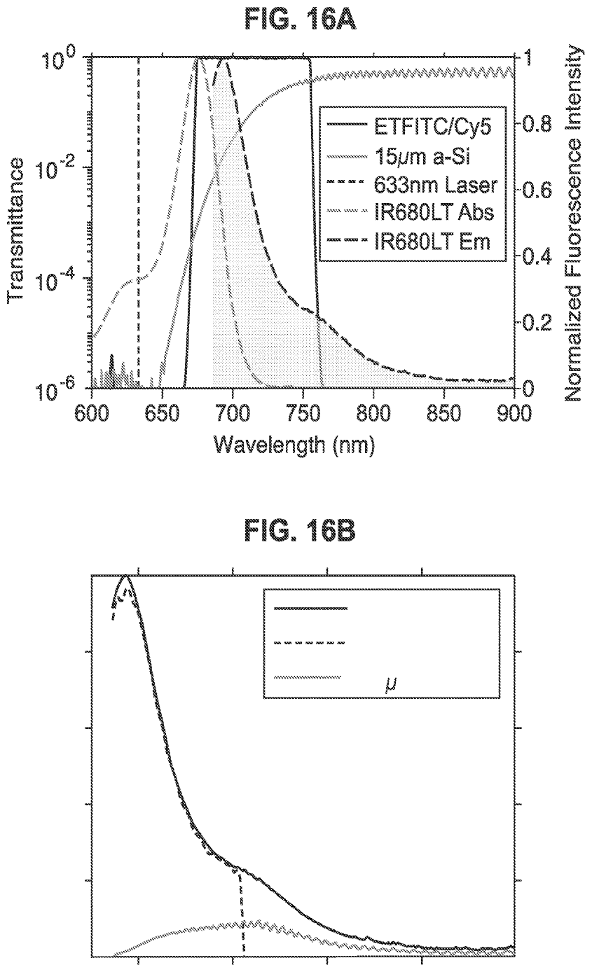

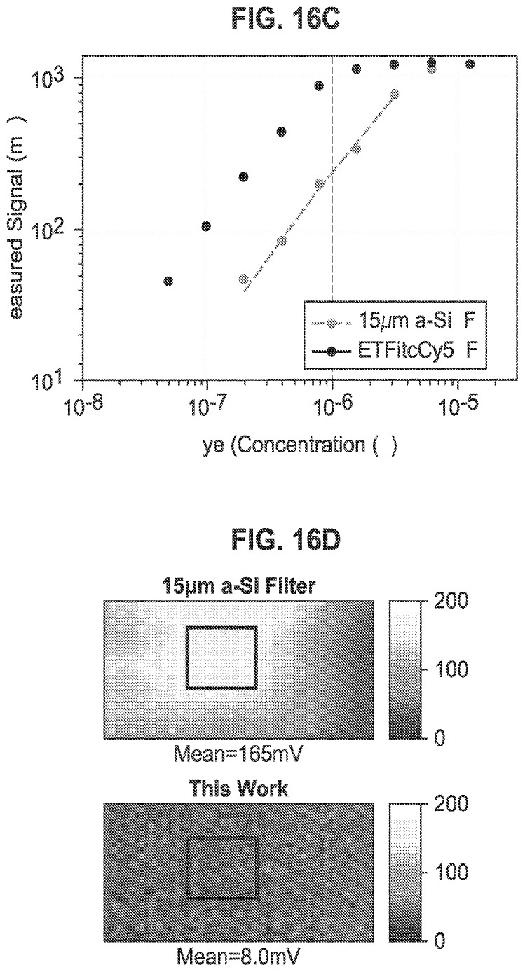

To demonstrate the performance improvements over absorption filters gained through this approach, we compare the proposed optical frontend with the 15 μm-thick amorphous silicon (a-Si) filter presented in prior work (FIGS. 16A-16D).

As observed in the measured optical transmittance spectra of both filters (FIG. 16A), the interference filter exhibits a more ideal filter characteristic with a sharper band-edge and higher pass-band transmittance, minimizing loss of fluorescence emissions. The total collected fluorescence emission is dependent on the collection efficiency of the imaging system, the absorption of the dye at the chosen excitation wavelength, and the overlap of the dye emission spectra with the filter passband. For the a-Si filter, the gradual roll-off in the filter response forces a significant trade-off between efficient excitation of the fluorophore and fluorescence emission collection. When the a-Si is used with IRDye 680LT, which has a Stoke's shift (19 nm) similar to many conventional fluorophores, the laser excitation may be at 633 nm for adequate excitation rejection, which is only at 33% of the absorption peak. At the emission peak (693 nm), the passband transmittance of the 15 μm-thick a-Si filter is just 1.6% and rises to maximum pass-band transmittance of 54% due to reflection losses from the high refractive index of the a-Si (4.3). On the other hand, with the proposed optical front-end, the laser excitation wavelength can be as close to 10 nm from the filter band-edge without significant excitation bleed-through (as demonstrated in FIG. 2g with the 488 nm laser excitation), allowing for both optimal excitation of the fluorophore and fluorescence emission collection. This fact is clear in the effective emission spectra of IRDye680LT for both filters (FIG. 16B), which is product of the dye emission spectra and the filter transmittance. The total fraction of emitted fluorescence passed by the a-Si filter is 4.24× less than that passed by the interference filter.

This result is confirmed by sensitivity measurements performed by imaging a 2× series dilution of IRDye680LT in 1×PBS with VISION using both the a-Si absorption filter and the ETFitcCy5 interference filter used in this work (FIG. 16C). Since the FOP trades-off improved resolution for reduced collection efficiency, for a fair comparison of filter performance that does not include this tradeoff, a 250 μm FOP is used with both filters. The dilution series is prepared by diluting a 12.5 μM stock solution of IRDye680LT NHS ester dissolved in 1×PBS by half until the concentration reaches 48.8 nM. 35 μL of each solution in the series is pipetted into chambered cover glass wells.

Further improvements in the fluorescence collection efficiency of the proposed optical front-end can be made by choosing an interference filter with a band-edge closer to the absorption edge of the dye to allow for excitation of 680LT at the absorption peak (676 nm).

The a-Si filter also suffers from reduced contrast due to increased background passed by the filter. Firstly, the a-Si inherently has a long-pass characteristic as opposed the bandpass characteristic of the interreference filter, such that additional out-of-band autofluorescence and ambient light is incident on the sensor. Secondly, compared to the proposed optical front-end, the a-Si filter provides significantly less excitation rejection. Images of the excitation bleed-through at 633 nm taken with an imager chip with each filter (FIG. 16D), shows a more than 20× increase in measured excitation background with the a-Si filter.

### Example 9: Imaging Chip Construction

The system described herein includes a composite optical front-end design that simultaneously addresses two optical design challenges in contact-imaging: multiplexed fluorescence filtering and high-resolution imaging. Opposed to previous approaches to on-chip optical front-end design which have focused on spectrally selective absorption filters, this system introduces an angle-selective low-NA FOP to compensate for the angle-sensitivity of multi-layer interference filters. The resulting composite filter maintains nearly 6 orders of magnitude excitation rejection across all AOIs while harnessing the inherent advantages of interference filters: unparalleled performance, versatile design for any visible or NIR fluorophore, and easy implementation of multicolor fluorescence imaging. Furthermore, this design may be composed entirely of commercially available optical components and utilizes no specialized fabrication steps. The FOP enables a more than 3× improvement in imager resolution over previously implemented on-chip collimating structures. This design includes a multiplexed fluorescence imaging on-chip. This innovation is relevant not only in the intraoperative imaging space, but also in a broad arena of biomedical applications where contact imagers show promise, such as functional brain imaging of free-moving animals, in vivo monitoring of immunotherapeutic response, and high-throughput molecular screening and diagnostics.

To emphasize clinical relevance for intraoperative surgical guidance, this disclosure shows that VISION, enabled by the optical front-end, can simultaneously identify microscopic tumor foci and nerves in resected prostate tissue. Moreover, the disclosure illustrates three clinical scenarios in surgical guidance: (i) detection of microscopic disease at the surgical margin, (ii) assessment of local spread of cancer into adjacent healthy tissue, and (iii) identification of a metastatic lymph node. These examples highlight the potential of this technology to both improve surgical success rates and minimize the adverse effects of treatment.

First, the interference filter can be fabricated directly on FOP, reducing the overall thickness of the optical front-end, improving the achievable resolution, and eliminating image artifacts. The filter should also be modified to cover NIR wavelengths to be compatible with most cancer- and nerve-specific intraoperative imaging agents currently in clinical trials. Next, imaging chips could be integrated with fiber-coupled excitation onto (or within) a laparoscopic probes for integration within robotic surgical workflows. Extensive animal studies demonstrating in vivo imaging with clinically used intraoperative agents are utilized to illustrate clinical feasibility. With these improvements, fluorescence imagers can play a critical role in intraoperative surgical guidance.

The following describes one particular process for fabricating an example imaging system in accordance with this disclosure. To the extent that particular values are indicated, these values are merely one example for the fabrication process, and this process can be repeated using alternate values for the various components and still achieve an imaging system, including an optical front-end with a FOP, that fall under the systems and techniques of this disclosure.

For filter fabrication, the surfaces of the chip, FOP, and interference filter are cleaned with 100% isopropyl alcohol. Next, optically transparent epoxy is mixed and placed in a vacuum chamber for 20 minutes to remove any bubbles. The interference filter is first epoxied with the filter side in direct contact with the chip and cured in a controlled temperature chamber (e.g., 45 minutes at 65° C.). After the filter is fully cured, the procedure is repeated to epoxy the FOP to the interference filter. Finally, to prevent optical leakage, the edges of the chip and filter are sealed with black epoxy and cured at 85° C. for 90 minutes.

Filter characterization measurements include angular transmittance measurements, which were performed using an optical power meter and fiber-coupled continuous lasers. The lasers, driven by a TEC-controlled driver, are collimated and aligned to be normally incident on the power meter. Narrow band interference filters for each laser line eliminate any out-of-band emissions. To calculate transmittance, the incident power is first measured without the sample present. Then, the sample is clamped into a motorized rotating stage to precisely measure the optical power transmitted through the filter at 1° increments. The transmittance at each measurement is taken as the fraction the total laser power that passes through the filter. To prevent any optical leakage from the environment, all measurements are performed in a light-proof box. Data for the transmittance spectra of the multi-bandpass interference filter is used.

To acquire images with VISION, a custom PCB is used to supply all power and biasing for the imaging chip as well as to read off and digitize each captured image frame. All timing and control signals necessary for image acquisition are generated by an FPGA, which is also used as digital interface between the imaging chip and a computer. A custom software GUI is used to visualize and capture image data on a laptop.

Resolution measurements are performed using a negative USAF-1951 resolution test target. The test target is coated one with Cy5.5 dye dissolved in DMSO and is sealed with a quartz coverslip. To verify even distribution of dye, a reference image of the target is taken on a benchtop microscope at 2.5× with an integration time of 1 s. The target is then placed dye-coated-side down directly on the imaging chip. Excitation light is provided by the aforementioned fiber-coupled and collimated 633 nm laser operated at approximately 1 mW. To minimize background, all imaging is performed in an optically isolated box. Each element is centered on the imager to minimize illumination variation and is imaged. 100 50 ms frames are captured for each imaging area and averaged to produce the final image. The contrast for each element is measured in ImageJ (NIH). Contrast is defined as [(I]_max−I_min) V (I_max+I_min-2I_background), where I_max is the maximum pixel value in the bright bars on the target, I_min is the minimum pixel value in the dark bars on the target, and I_background is the average pixel value when the excitation source is off.

The paraffin-embedded tissue blocks for each sample are sectioned at 4 μm and mounted onto glass slides. One representative slide from each block is stained with hemoxylin and eosin (H&E) for histological analysis. The remaining slides are used for immunofluorescence staining. To label prostate tumor, an anti-PSMA rabbit primary antibody is used with an IRDye 680LT goat anti-rabbit secondary antibody (LI-COR). For nerve labeling, an anti-S100 mouse primary antibody is used with an Alexa Fluor 488 anti-mouse secondary antibody (Invitrogen). As a reference image and to verify staining, full-slide fluorescence scans of 1 representative slide from each block are acquired with a fluorescence microscope using a 10× objective. Both immunofluorescence and histological images are compared by a trained pathologist to verify specific staining of tumor and nerves. The remaining immunofluorescence slides are used for imaging experiments.

When imaging ex vivo tissue samples, an inverted fluorescence microscope is used for sample alignment with the imaging chip and to acquire reference images. The sample slide is placed in the microscope slide mount with the coverslip facing away from the objective lens. With the microscope, the imaging area of interest is identified and centered over the objective. The imaging chip is then inverted and placed on the coverslip such that it is imaging the same sample area as the microscope. To align the microscope objective and imaging chip, an excitation wavelength in the passband of the chip is shined through a high-magnification objective and onto the chip. The chip position is adjusted such that the excitation light is centered within the FOV of the imager. The aforementioned collimated, fiber-coupled lasers are used to provide fluorescence excitation at 488 nm and 633 nm and are both operated at 15 mW. The excitation light is introduced at the back of the slide and is incident on the chip at approximately 70° with the beam parallel to the long dimension of the chip. For each region of interest on the sample, separate microscope and chip images are acquired with each excitation wavelength. Microscope images are captured using a 2.5× objective and 5 s exposure time. For the chip, 100 images are acquired at each excitation wavelength with 50 ms (488 nm) and 75 ms (633 nm) integration times. Image post-processing is performed in ImageJ. Raw images are averaged, thresholder=d, and then colored. Multiplexed images are created by overlaying separate images from each fluorescence channel.

For purposes of completeness, various aspects of the present disclosure are set out in the following numbered clauses.
Aspect 1. A system for fluorescence imaging, comprising:
   an optical front-end comprising a first layer of an optical filter and a second layer of a collimator.
Aspect 2. The system of Aspect 1, wherein the optical front-end comprises a plurality of layers of alternating layers of materials with different refractive indices to create a long pass, short pass, band-pass or multiband pass interreference filter.
Aspect 3. The system of Aspect 2, wherein the plurality of layers further includes an absorption filter.
Aspect 4. The system of any one of Aspects 1-3, wherein the optical front-end is affixed to an imaging sensor comprising an array of pixel and wherein the system further comprises a visualization system in electrical communication with the imager.
Aspect 5. The system of Aspect 4, wherein the imaging sensor is a CMOS sensor.
Aspect 6. The system of any one or more of Aspects 1-5, wherein a thickness of the optical front-end is less than or equal to 5 mm.
Aspect 7. The system of any one or more of Aspects 1-6, wherein the thickness of the optical front-end is less than or equal to 2.5 mm.
Aspect 8. The system of any one or more of Aspects 1-7, wherein the thickness of the optical front-end is less than or equal to 1.25 mm.
Aspect 9. The system of any one or more of Aspects 1-8, wherein the thickness of the optical front-end is less than or equal to 500 μm.
Aspect 10. The system of any one or more of Aspects 1-9, wherein the thickness of the optical front-end is less than or equal to 250 μm.
Aspect 11. The system of any one or more of Aspects 1-10, wherein the thickness of the optical front-end is less than or equal to 150 μm
Aspect 12. The system of any one or more of Aspects 1-11, wherein the collimator is angle-selective.
Aspect 13. The system of Aspect 12, wherein the collimator further has one of:
   2 orders of magnitude rejection at 30 degrees or greater off axis,
   3 orders of magnitude rejection at 30 degrees or greater off axis,
   4 orders of magnitude rejection at 30 degrees or greater off axis,
   5 orders of magnitude rejection at 30 degrees or greater off axis, or
   6 orders of magnitude or greater rejection at 30 degrees or greater off axis.
Aspect 14. The system of any one or more of 31-112, wherein the collimator is a parallel-hole collimator.
Aspect 15. The system of any one or more of Aspects 1-14, wherein the collimator is a fiber optic plate.
Aspect 16. The system of any one or more of Aspects 1-15, wherein the collimator comprises an absorptive material surrounding an array of optical fibers.
Aspect 17. The system of any one or more of Aspects 1-15, wherein the collimator is an absorptive material surrounding one or more holes filled with a transparent material.
Aspect 18. The system of any one or more of Aspects 1-15, wherein the collimator is an absorptive material surrounding holes that are either filled with air or in a vacuum.
Aspect 19. The system of any one or more of Aspects 1-18, wherein the imager is operationally integrated with a surgical tool.
Aspect 20. The system of Aspect 19, wherein the surgical tool is a periscopic probe or laparoscopic robotic instrument.
Aspect 21. The system of any one or more of Aspects 1-20, wherein the imager comprises at least one LED or at least one laser diode light source.
Aspect 22. The system of any one or more of Aspects 1-18 or 21, wherein the imager is operationally integrated with an implantable imager.
Aspect 23. The system of any one or more of Aspects 1-18 or 21, wherein the imager is operationally integrated with a lab-on a chip.
Aspect 24. The system of Aspect 23, wherein the lab on chip is a microarray of DNA, RNA, proteins, cells, tissue, or any biological sample.
Aspect 25. The system of Aspect 23, wherein the lab on chip is a diagnostic assay, including a PCR test, ELISA, or lateral flow assay.
Aspect 26. The system of any one or more of Aspects 23-25, wherein the imager comprises at least one LED or at least one laser diode light source.
Aspect 27. The system of any one or more of Aspects 1-26, wherein the system is lens-free.
Aspect 28. The system of any one or more of Aspects 1-27, wherein the system utilizes machine learning to improve image quality.
Aspect 29. The system of any one or more of Aspects 1-28, wherein a sample being imaged by the system comprises one or more of diseased tissue and cancerous tissue.
Aspect 30. The system of any one or more of Aspects 1-29, wherein a labeling agent being imaged comprises one or more of an antibody, an antibody mimetic, a peptide, a peptoid, an aptamer, or a small molecule ligand that selectively binds to the cellular, protein, DNA, RNA, molecular or chemical marker of interest.
Aspect 31. The system of Aspect 30, wherein the cellular marker of interest comprises one or more of a tumor-specific antigen, a tumor-associated antigen, and an immune activation marker.
Aspect 32. The system of any one or more of Aspects 1-31, wherein a sample is imaged with at least two different fluorophore conjugates, wherein each fluorophore conjugate comprises a different fluorophore that emits fluorescent light at a different emission wavelength, and wherein each fluorophore conjugate comprises a different binding agent that selectively binds to a different marker.
Aspect 33. A method for imaging a biological sample, comprising:
   applying a marker to a tissue; and
   obtaining an image of the tissue using a system as described in any of Aspects 1-31.
Aspect 34. The method of Aspect 33, further comprising resecting the tissue to remove diseased tissue from the marked tissue.
Aspect 35. The method of any of Aspects 33-34, wherein the marker is a fluorescent dye selected from SYBR green, SYBR gold, CAL Fluor Gold 540, CAL Fluor Orange 560, CAL Fluor Red 590, CAL Fluor Red 610, CAL Fluor Red 635, Quasar 570, Quasar 670, Quasar 705, fluorescein, fluorescein isothiocyanate, hexachlorofluorescein, 4′,6-diamidino-2-phenylindole, Hoechst, rhodamine, carboxy-X-rhodamine, and combinations thereof.
Aspect 36. The method of any of Aspects 33-34, wherein the marker is a fluorescent probe comprising a binding agent and a fluorophore.
Aspect 37. The method of Aspect 36, wherein the fluorophore is selected from Alexa Fluor 488, Alexa Fluor 546, Alexa Fluor 555, Alexa Fluor 594, Alexa Fluor 647, Alexa Fluor 784, Cy3, Cy3.5, Cy5, Cy5.5, and Cy7.
Aspect 38. The method of Aspect 36, wherein the binding moiety is selected from a carbohydrate, a lipid, a peptide, a nucleic acid, a protein, and a small molecule.
Aspect 39. The method of any of Aspects 33-38 further comprising illuminating the tissue with a light source.
Aspect 40 The method of any of Aspects 33-39, wherein obtaining an image of the tissue comprises contacting the optical front-end to the tissue.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. A system for fluorescence imaging, comprising:
an optical front-end comprising a first layer of an optical filter and a second layer of a collimator.

2. The system of claim 1, wherein the optical front-end comprises a plurality of layers of alternating layers of materials with different refractive indices to create a long pass, short pass, band-pass or multiband pass interference filter.

3. The system of claim 2, wherein the plurality of layers further includes an absorption filter.

4. The system of any one of claims 1-3, wherein the optical front-end is affixed to an imaging sensor comprising an array of pixels and wherein the system further comprises a visualizations system in electrical communication with the imaging chip.

5. The system of claim 4, wherein the imaging sensor is a CMOS sensor.

6. The system of any one or more of claims 1-5, wherein a thickness of the optical front-end is less than or equal to 5 mm.

7. The system of any one or more of claims 1-6, wherein the thickness of the optical front-end is less than or equal to 2.5 mm.

8. The system of any one or more of claims 1-7, wherein the thickness of the optical front-end is less than or equal to 1.25 mm.

9. The system of any one or more of claims 1-8, wherein the thickness of the optical front-end is less than or equal to 500 μm.

10. The system of any one or more of claims 1-9, wherein the thickness of the optical front-end is less than or equal to 250 μm.

11. The system of any one or more of claims 1-10, wherein the thickness of the optical front-end is less than or equal to 150 μm

12. The system of any one or more of claims 1-11, wherein the collimator is angle-selective.

13. The system of claim 12, wherein the collimator further has one of:
2 orders of magnitude rejection at 30 degrees or greater off axis,
3 orders of magnitude rejection at 30 degrees or greater off axis,
4 orders of magnitude rejection at 30 degrees or greater off axis,
5 orders of magnitude rejection at 30 degrees or greater off axis, or
6 orders of magnitude or greater rejection at 30 degrees or greater off axis.

14. The system of any one or more of 31-112, wherein the collimator is a parallel-hole collimator.

15. The system of any one or more of claims 1-14, wherein the collimator is a fiber optic plate.

16. The system of any one or more of claims 1-15, wherein the collimator comprises an absorptive material surrounding an array of optical fibers.

17. The system of any one or more of claims 1-15, wherein the collimator is an absorptive material surrounding one or more holes filled with a transparent material.

18. The system of any one or more of claims 1-15, wherein the collimator is an absorptive material surrounding holes that are either filled with air or in a vacuum.

19. The system of any one or more of claims 1-18, wherein the imaging chip is operationally integrated with a surgical tool.

20. The system of claim 19, wherein the surgical tool is a periscopic probe or laparoscopic robotic instrument.

21. The system of any one or more of claims 1-20, wherein the imaging chip comprises at least one LED or at least one laser diode light source.

22. The system of any one or more of claim 1-18 or 21, wherein the imaging chip is operationally integrated with an implantable imager.

23. The system of any one or more of claim 1-18 or 21, wherein the imaging chip is operationally integrated with a lab-on a chip.

24. The system of claim 23, wherein the lab on chip is a microarray of DNA, RNA, proteins, cells, tissue, or any biological sample.

25. The system of claim 23, wherein the lab on chip is a diagnostic assay, including a PCR test, ELISA, or lateral flow assay.

26. The system of any one or more of claims 23-25, wherein the imaging chip comprises at least one LED or at least one laser diode light source.

27. The system of any one or more of claims 1-26, wherein the system is lens-free.

28. The system of any one or more of claims 1-27, wherein the system utilizes machine learning to improve image quality.

29. The system of any one or more of claims 1-28, wherein a sample being imaged by the system comprises one or more of diseased tissue and cancerous tissue.

30. The system of any one or more of claims 1-29, wherein a labeling agent being imaged comprises one or more of an antibody, an antibody mimetic, a peptide, a peptoid, an aptamer, or a small molecule ligand that selectively binds to the cellular, protein, DNA, RNA, molecular or chemical marker of interest.

31. The system of claim 30, wherein the cellular marker of interest comprises one or more of a tumor-specific antigen, a tumor-associated antigen, and an immune activation marker.

32. The system of any one or more of claims 1-31, wherein a sample is imaged with at least two different fluorophore conjugates, wherein each fluorophore conjugate comprises a different fluorophore that emits fluorescent light at a different emission wavelength, and wherein each fluorophore conjugate comprises a different binding agent that selectively binds to a different marker.

33. A method for imaging a biological sample, comprising:
applying a marker to a tissue; and
obtaining an image of the tissue using a system as described in any of claims 1-31.

34. The method of claim 33, further comprising resecting the tissue to remove diseased tissue from the marked tissue.

35. The method of any of claims 33-34, wherein the marker is a fluorescent dye selected from SYBR green, SYBR gold, CAL Fluor Gold 540, CAL Fluor Orange 560, CAL Fluor Red 590, CAL Fluor Red 610, CAL Fluor Red 635, Quasar 570, Quasar 670, Quasar 705, fluorescein, fluorescein isothiocyanate, hexachlorofluorescein, 4′,6-diamidino-2-phenylindole, Hoechst, rhodamine, carboxy-X-rhodamine, and combinations thereof.

36. The method of any of claims 33-34, wherein the marker is a fluorescent probe comprising a binding agent and a fluorophore.

37. The method of claim 36, wherein the fluorophore is selected from Alexa Fluor 488, Alexa Fluor 546, Alexa Fluor 555, Alexa Fluor 594, Alexa Fluor 647, Alexa Fluor 784, Cy3, Cy3.5, Cy5, Cy5.5, and Cy7.

38. The method of claim 36, wherein the binding moiety is selected from a carbohydrate, a lipid, a peptide, a nucleic acid, a protein, and a small molecule.

39. The method of any of claims 33-38 further comprising illuminating the tissue with a light source.

40. The method of any of claims 33-39, wherein obtaining an image of the tissue comprises contacting the optical front-end to the tissue.
